(19) United States
(12) Patent Application Publication
(10) Pub. No.: US 2005/0070527 A1
(43) Pub. Date: Mar. 31, 2005

(54) Quinoxaline compounds

(76) Inventors: James P. Edwards, San Diego, CA (US); Jennifer D. Venable, Solana Beach, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/953,126

(22) Filed: Sep. 29, 2004

Related U.S. Application Data
(60) Provisional application No. 60/507,176, filed on Sep. 30, 2003.

(51) Int. Cl. A61K 31/551; A61K 31/498; C07D 487/02

(52) U.S. Cl. 514/218; 514/249; 540/575; 544/350; 544/354

(57) ABSTRACT

Quinoxaline compounds, compositions, methods of making them, and methods of using them in leukocyte recruitment inhibition, in modulating an H₄ receptor, and in treating conditions such as inflammation, H₄ receptor-mediated conditions, and related conditions.

### FIELD OF THE INVENTION

The invention relates to novel, pharmaceutically active, fused heterocyclic compounds, more particularly quinoxaline compounds, and methods of using them to treat or prevent disorders and conditions mediated by the histamine H₄ receptor.

### BACKGROUND OF THE INVENTION

Histamine was first identified as a hormone (G. Barger and H. H. Dale, J. Physiol. (London) 1910, 41:19-59) and has since been demonstrated to play a major role in a variety of physiological processes, including the inflammatory “triple response” via H₃ receptors (A. S. F. Ash and H. O. Schild, Br. J. Pharmac. Chemother. 1966, 27:427-439), gastric acid secretion via H₂ receptors (J. W. Black et al., Nature 1972, 236:385-390), and neurotransmitter release in the central nervous system via H₃ receptors (J.-M. Arrang et al., Nature 1983, 302:832-837) (for review see S. J. Hill et al., Pharmacol. Rev. 1997, 49(3):253-278). All three histamine receptor subtypes have been demonstrated to be members of the superfamily of G protein-coupled receptors (I. Gantz et al., Proc. Natl. Acad. Sci. U.S.A. 1991, 88:429-433; T. W. Lovenberg et al., Mol. Pharmacol. 1999, 55(6):1101-1107; M. Yamashita et al., Proc. Natl. Acad. Sci. U.S.A. 1991, 88:11515-11519). There are, however, additional functions of histamine that have been reported, for which no receptor has been identified. For example, in 1994, Raible et al., demonstrated that histamine and R-α-methylhistamine could activate calcium mobilization in human eosinophils (D. G. Raible et al., Am. J. Respir. Crit. Care Med. 1994, 149:1506-1511). These responses were blocked by the H₃-receptor antagonist thioperamide. However, R-α-methylhistamine was significantly less potent than histamine, which was not consistent with the involvement of known H₃ receptor subtypes. Therefore, Raible et al., hypothesized the existence of a novel histamine receptor on eosinophils that was non-H₁, non-H₂, and non-H₃. Most recently several groups (T. Oda et al., J. Biol. Chem. 2000, 275(47):36781-36786; C. Liu et al., Mol. Pharmacol. 2001, 59(3):420-426; T. Nguyen et al., Mol. Pharmacol. 2001, 59(3):427-433; Y. Zhu et al., Mol. Pharmacol. 2001, 59(3):434-441; K. L. Morse et al., J. Pharmacol. Exp. Ther. 2001, 296(3):1058-1066) have identified and characterized a fourth histamine receptor subtype, the H₄ receptor. This receptor is a 390 amino acid, seven-transmembrane, G protein-coupled receptor with approximately 40% homology to the histamine H₃ receptor. In contrast to the H₃ receptor, which is primarily located in the brain, the H₄ receptor is expressed at greater levels in eosinophils and mast cells, among other cells, as reported by Liu et al. (see above) and C. L. Hofstra et al. (J. Pharmacol. Exp. Ther. 2003, 305(3):1212-1221). Because of its preferential expression on immunocompetent cells, this H₄ receptor is closely related with the regulatory functions of histamine during the immune response.

A biological activity of histamine in the context of immunology and autoimmune diseases is closely related with the allergic response and its deleterious effects, such as inflammation. Events that elicit the inflammatory response include physical stimulation (including trauma), chemical stimulation, infection, and invasion by a foreign body. The inflammatory response is characterized by pain, increased temperature, redness, swelling, reduced function, or a combination of these.

Mast-cell de-granulation (exocytosis) releases histamine and leads to an inflammatory response that may be initially characterized by a histamine-modulated wheal and flare reaction. A wide variety of immunological stimuli (e.g., allergens or antibodies) and non-immunological (e.g., chemical) stimuli may cause the activation, recruitment, and de-granulation of mast cells. Mast-cell activation initiates allergic (H₁) inflammatory responses, which in turn cause the recruitment of other effector cells that further contribute to the inflammatory response. The histamine H₂ receptors modulate gastric acid secretion, and the histamine H₃ receptors affect neurotransmitter release in the central nervous system.

Modulation of H₄ receptors controls the release of inflammatory mediators and inhibits leukocyte recruitment, thus providing the ability to prevent and/or treat H₄-mediated diseases and conditions, including the deleterious effects of allergic responses such as inflammation. Compounds according to the present invention have H₄ receptor modulating properties. Compounds according to the present invention have leukocyte recruitment inhibiting properties. Compounds according to the present invention have anti-inflammatory properties.

Examples of textbooks on the subject of inflammation include J. I. Gallin and R. Snyderman, Inflammation: Basic Principles and Clinical Correlates, 3^{rd} Edition, (Lippincott Williams & Wilkins, Philadelphia, 1999); V. Stvrtinova, J. Jakubovsky and 1. Hulin, “Inflammation and Fever”, Pathophysiology Principles of Diseases (Textbook for Medical Students, Academic Press, 1995); Cecil et al., Textbook Of Medicine, 18^{th} Edition (W. B. Saunders Company, 1988); and Steadmans Medical Dictionary.

Background and review material on inflammation and conditions related with inflammation can be found in articles such as the following: C. Nathan, Points of control in inflammation, Nature 2002, 420:846-852; K. J. Tracey, The inflammatory reflex, Nature 2002, 420:853-859; L. M. Coussens and Z. Werb, Inflammation and cancer, Nature 2002, 420:860-867; P. Libby, Inflammation in atherosclerosis, Nature 2002, 420:868-874; C. Benoist and D. Mathis, Mast cells in autoimmune disease, Nature 2002, 420:875-878; H. L. Weiner and D. J. Selkoe, Inflammation and therapeutic vaccination in CNS diseases, Nature 2002, 420:879-884; J. Cohen, The immunopathogenesis of sepsis, Nature 2002, 420:885-891; D. Steinberg, Atherogenesis in perspective: Hypercholesterolemia and inflammation as partners in crime, Nature Medicine 2002, 8(11):1211-1217.

Inflammation herein refers to the response that develops as a consequence of histamine release, which in turn is caused by at least one stimulus. Examples of such stimuli are immunological stimuli and non-immunological stimuli.

Inflammation is due to any one of a plurality of conditions such as allergy, asthma, chronic obstructed pulmonary disease (COPD), atherosclerosis, rheumatoid arthritis, multiple sclerosis, inflammatory bowel diseases (including Crohn's disease and ulcerative colitis), psoriasis, allergic rhinitis, scleroderma, autoimmune thyroid diseases, immune-mediated (also known as type 1) diabetes mellitus and lupus, which are characterized by excessive or prolonged inflammation at some stage of the disease. Other autoimmune diseases that lead to inflammation include Myasthenia gravis, autoimmune neuropathies, such as Guillain-Barré, autoimmune uveitis, autoimmune hemolytic anemia, pernicious anemia, autoimmune thrombocytopenia, temporal arteritis, anti-phospholipid syndrome, vasculitides, such as Wegener's granulomatosis, Behcet's disease, dermatitis herpetiformis, pemphigus vulgaris, vitiligio, primary biliary cirrhosis, autoimmune hepatitis, autoimmune oophoritis and orchitis, autoimmune disease of the adrenal gland, polymyositis, dermatomyositis, spondyloarthropathies, such as ankylosing spondylitis, and Sjogren's syndrome. Regarding the onset and evolution of inflammation, inflammatory diseases or inflammation-mediated diseases or conditions include, but are not limited to, acute inflammation, allergic inflammation, and chronic inflammation.

Cited references are incorporated herein by reference.

### SUMMARY OF THE INVENTION

The invention features a compound of formula (I): 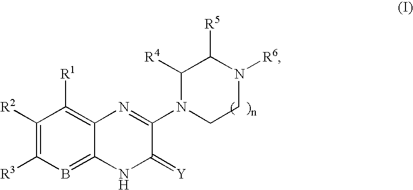
wherein
B is, independently from other member and substituent assignments, N or CR⁷;
Y is, independently from other member and substituent assignments, O, S or NH;
n is, independently from member and substituent assignments, 1 or 2;
each of the substituents R¹⁻³ and R⁷ is, independently from other member and substituent assignments, H, F, Cl, Br, I, C₁₋₄ alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of R^{a}, and R^{b} is, independently from other substituent assignments, selected from H, C₁₋₄alkyl, phenyl, benzyl or phenethyl, and wherein any phenyl, alkyl, and cycloalkyl moiety in any of said R¹⁻³, R⁷, R^{a}, and R^{b}, is optionally, and independently from other substituent assignments, substituted with 1 to 3 substituents selected from C₁₋₃alkyl, halo, hydroxy, amino, and C₁₋₃alkoxy;
each of R⁴ and R⁵ is, independently from other member and substituent assignments, H or C₁₋₃alkyl;
R⁶ is, independently from other member and substituent assignments, H, C₁₋₆ alkyl, C₃₋₅alkenyl with no sp²-carbon member attached directly to the R⁶-attached nitrogen member, C₃₋₅alkynyl with no sp-carbon member attached directly to the R⁶-attached nitrogen member, CH₂CH₂OH, or —C₁₋₄alkyl-O—C₁₋₄alkyl;
   alternatively, R⁶ may be taken together with R⁵, the R⁵-attached carbon member, and the R⁶-attached nitrogen member to form a 5-, 6- or 7-membered heterocyclic ring HetCyc1, wherein said ring HetCyc1 has 0 or 1 additional heteroatoms selected from O, S, >NH or >NC₁₋₆alkyl, and wherein said ring HetCyc1 is substituted with 0, 1, 2 or 3 substituents each selected independently from other substituent assignments from C₁₋₃alkyl, halo, hydroxy, amino, and C₁₋₃alkoxy;
an enantiomer, diastereomer, racemate thereof, or a pharmaceutically acceptable salt or ester thereof.

Also, one of ordinary skill in the art will recognize that compounds according to this invention may exist in tautomeric forms. When this is the case, reference herein to one tautomeric form is meant to refer to at least one of such tautomeric forms, and all such tautomeric forms are within the scope of this invention. For example, compounds of formula (I) may exist in their tautomeric forms, represented by formula (II):

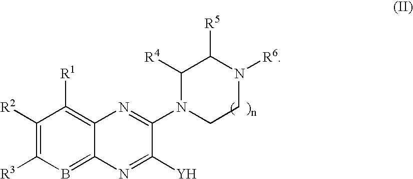

Such compounds of formula (II) are also encompassed within the present invention.

Similarly, isomeric forms of the compounds of formula (I), and of their pharmaceutically acceptable salts and esters, are encompassed within the present invention, and reference herein to one of such isomeric forms is meant to refer to at least one of such isomeric forms. One of ordinary skill in the art will recognize that compounds according to this invention may exist, for example in a single isomeric form whereas other compounds may exist in the form of a regioisomeric mixture.

Whether stated explicitly or not in any part of the written description and claims, it is understood that each substituent and member assignment in the context of this invention is made independently of any other member and substituent assignment, unless stated otherwise. By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S¹ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent R assignments described herein. The foregoing convention given herein for substituents extends, when applicable, to members such as X, Y, Z, and W, and the index n.

Furthermore, when more than one assignment is given for any member or substituent, embodiments of this invention comprise the various groupings that can be made from the listed assignments and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent R assignments described herein. The foregoing convention given herein for substituents extends, when applicable, to members such as X, Y, Z, and W, and the index n.

The nomenclature “Cᵢ₋ⱼ” with j>i, when applied herein to a class of substituents, is meant to refer to embodiments of this invention for which each and every one of the number of carbon members, from i to j including i and j, is realized. By way of example, the term C₁₋₃ refers independently to embodiments that have one carbon member (Cl), embodiments that have two carbon members (C₂), and embodiments that have three carbon members (C₃).

When any variable referring to a substituent, compound member or index, occurs more than once, the full range of assignments is meant to apply to each occurrence, independently of the specific assignment(s) to any other occurrence of such variable.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

The invention also features a pharmaceutical composition for treating or preventing an H₄ receptor-mediated condition in a subject, comprising a therapeutically effective amount for treating or preventing an H₄ receptor-mediated condition of at least one of an H₄ receptor modulator of formula (I), an enantiomer, diastereomer, racemate thereof, and pharmaceutically acceptable salt and ester thereof. In addition, the invention features a pharmaceutical composition for inhibiting leukocyte recruitment in a subject, comprising a therapeutically effective amount for inhibiting leukocyte recruitment in a subject of at least one of a leukocyte recruitment inhibitor of formula (I), an enantiomer, diastereomer, racemate thereof, and pharmaceutically acceptable salt and ester thereof. The invention additionally features an anti-inflammatory composition, comprising a therapeutically effective amount for treating or preventing inflammation of at least one of an anti-inflammatory compound of formula (I), an enantiomer, diastereomer, racemate thereof, and pharmaceutically acceptable salt and ester thereof.

The invention features methods for treating or preventing inflammation in a subject, comprising administering to the subject in connection with an inflammatory response a pharmaceutical composition that comprises a therapeutically effective amount of at least one of an anti-inflammatory compound of formula (I), an enantiomer, diastereomer, racemate thereof, and pharmaceutically acceptable salt and ester thereof. The invention also features methods for treating or preventing an H₄ receptor-mediated condition in a subject, comprising administering to the subject a pharmaceutical composition that comprises a therapeutically effective amount of at least one of an H₄ receptor modulator of formula (I), an enantiomer, diastereomer, racemate thereof, and pharmaceutically acceptable salt and ester thereof. In addition, the invention features methods for modulating an H₄ receptor, comprising exposing an H₄ receptor to at least one of a compound of formula (I), an enantiomer, diastereomer, racemate thereof, and pharmaceutically acceptable salt and ester thereof. Furthermore, the invention features methods for inhibiting leukocyte recruitment in a subject, comprising administering to the subject a pharmaceutical composition that comprises a therapeutically effective amount of at least one of a leukocyte recruitment inhibitor of formula (I), an enantiomer, diastereomer, racemate thereof, and pharmaceutically acceptable salt and ester thereof.

The invention features a method for making a quinoxaline compound, such as a compound of formula (I), or an enantiomer, diastereomer, racemate thereof, or pharmaceutically acceptable salt or ester thereof, comprising reacting a diamino compound of formula (III) with an ester of formula (IV), wherein the meanings of R¹⁻³, and B are as described hereinabove, and R is one of C₁₋₆alkyl and benzyl. Structures of compounds of formulae (III) and (IV) are given as follows:

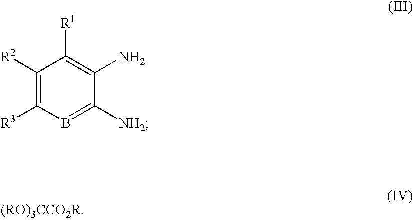

### DETAILED DESCRIPTION

The present invention is directed to compounds of formula (I) as herein defined, pharmaceutical compositions that contain at least one compound of formula (I), methods of using, including treatment and/or prevention of conditions such as those that are mediated by the H₄ receptor, and methods of making such compounds and pharmaceutical compositions.

The following terms are defined below, and by their usage throughout the disclosure.

“Alkyl” includes straight chain and branched hydrocarbons with at least one hydrogen removed to form a radical group. Alkyl groups include methyl, ethyl, propyl, isopropyl, butyl, isobutyl, t-butyl, 1-methylpropyl, pentyl, isopentyl, sec-pentyl, hexyl, heptyl, octyl, and so on. Alkyl does not include cycloalkyl.

“Alkenyl” includes straight chain and branched hydrocarbon radicals as above with at least one carbon-carbon double bond (sp²). Unless indicated otherwise by the prefix that indicates the number of carbon members, alkenyls include ethenyl (or vinyl), prop-1-enyl, prop-2-enyl (or allyl), isopropenyl (or 1-methylvinyl), but-1-enyl, but-2-enyl, butadienyls, pentenyls, hexa-2,4-dienyl, and so on. Alkenyl does not include cycloalkenyl.

“Alkynyl” includes straight chain and branched hydrocarbon radicals as above with at least one carbon-carbon triple bond (sp). Unless indicated otherwise by the prefix that indicates the number of carbon members, alkynyls include ethynyl, propynyls, butynyls, and pentynyls. Hydrocarbon radicals having a mixture of double bonds and triple bonds, such as 2-penten-4-ynyl, are grouped as akynyls herein.

“Alkoxy” includes a straight chain or branched alkyl group with a terminal oxygen linking the alkyl group to the rest of the molecule. Alkoxy includes methoxy, ethoxy, propoxy, isopropoxy, butoxy, t-butoxy, pentoxy and so on.

“Aminoalkyl”, “thioalkyl”, and “sulfonylalkyl” are analogous to alkoxy, replacing the terminal oxygen atom of alkoxy with, respectively, NH (or NR), S, and SO₂.

Unless indicated otherwise by the prefix that indicates the number of carbon members, “cycloalkyl” includes cyclopropyl, cyclobutyl, cyclopentyl, cyclohexyl, cycloheptyl, cyclooctyl, and so on.

Unless indicated otherwise by the prefix that indicates the number of members in the cyclic structure, “heterocyclyl” or “heterocycle” is a 3- to 8-member aromatic, saturated, or partially saturated single or fused ring system that comprises carbon atoms wherein the heteroatoms are selected, unless otherwise indicated, from N, O, and S. Examples of heterocyclyls include thiazoylyl, furyl, pyranyl, isobenzofuranyl, pyrrolyl, imidazolyl, pyrazolyl, isothiazolyl, isoxazolyl, pyridyl, pyrazinyl, pyrimidinyl, pyridazinyl, indolizinyl, isoindolyl, indolyl, indazolyl, purinyl, quinolyl, furazanyl, pyrrolidinyl, pyrrolinyl, imidazolidinyl, imidazolinyl, pyrazolidinyl, pyrazolinyl, piperidyl, piperazinyl, indolinyl, and morpholinyl. For example, preferred heterocyclyls or heterocyclic radicals include morpholinyl, piperazinyl, pyrrolidinyl, pyridyl, cyclohexylimino, cycloheptylimino, and more preferably, piperidyl.

“Halo” includes fluoro, chloro, bromo, and iodo, and is preferably fluoro or chloro.

As in standard chemical nomenclature, the group phenyl is herein referred to as “phenyl” or as “Ph”.

“Patient” or “subject” includes mammals such as human beings and animals (e.g., dogs, cats, horses, rats, rabbits, mice, non-human primates) in need of observation, experiment, treatment or prevention in connection with the relevant disease or condition. Preferably, the patient is a human being.

“Composition” includes a product comprising the specified ingredients in the specified amounts, including in the effective amounts, as well as any product that results directly or indirectly from combinations of the specified ingredients in the specified amounts.

“Therapeutically effective amount” or “effective amount” and grammatically related terms mean that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Table of Acronyms

| Term | Acronym |
|---|---|
| Tetrahydrofuran | THF |
| N,N-Dimethylformamide | DMF |
| N,N-Dimethylacetamide | DMA |
| Dimethyl sulfoxide | DMSO |
| tert-Butylcarbamoyl | BOC |
| Bovine serum albumin | BSA |
| High-pressure liquid chromatography | HPLC |
| Thin layer chromatography | TLC |

Particular preferred compounds of the invention comprise a quinoxaline compound of formula (I), or an enantiomer, diastereomer, racemate thereof, or a pharmaceutically acceptable salt or ester thereof, wherein R¹⁻⁶, B, Y, and n have any of the meanings defined hereinabove and equivalents thereof, or at least one of the following assignments and equivalents thereof. Such assignments may be used where appropriate with any of the definitions, claims or embodiments defined herein:
B is CR⁷;
Y is O;
n is 1;
each of R¹⁻³ and R⁷ is, independently from other member and substituent assignments, selected from the group consisting of H, —F, —Cl, —Br, —I, —CH₃, —CH₂CH₃, —OCH₃, —OCH₂CH₃, —OCH(CH₃)₂, cyclopropyl, cyclobutyl, cyclopentyl, cyclohexyl, —Ocyclopentyl, —Ocyclohexyl, —CF₃, —OCF₃, —SCF₃, —OH, —NO₂, —NH₂, —NHCH₃, —N(CH₃)₂, —N(CH₂CH₃)₂, —CN and phenyl;
more preferably, R¹⁻³ and R⁷ are, independently, selected from the group consisting of hydrogen, methyl, trifluoromethyl, methoxy, trifluoromethoxy, nitro, chloro, and fluoro. Further, it is most preferred that one or two of R¹⁻³ and R⁷ are not hydrogen;
R⁴ and R⁵ are independently selected from the group consisting of
a) H, and
b) —CH₃, —CH₂CH₃, —CH₂CH₂CH₃, —CH(CH₃)₂, n-butyl, i-butyl, and t-butyl;
more preferably, R⁴ and R⁵ are, independently, H or —CH₃;
R⁶ is selected from the group consisting of
a) H,
b) CH₂CH₂OH, and
c) —CH₃, —CH₂CH₃, —CH₂CH₂CH₃, —CH(CH₃)₂, n-butyl, i-butyl, t-butyl, —CH₂CH₂OCH₃, —CH₂CH₂OCH₂CH₃, —CH₂CH₂OCH₂CH₂CH₃, —CH₂CH₂OCH(CH₃)₂, —CH₂CH₂O-n-butyl, —CH₂CH₂O-1-butyl, and —CH₂CH₂O-t-butyl;
more preferably, R⁶ is selected from the group consisting of H, —CH₃ and —CH₂CH₃;
preferred R taken together with an adjacent R⁵ as well as their carbon and nitrogen of attachment is one of pyrrolidin-1,2-yl, pyrazolidin-1,5-yl, piperidin-1,2-yl, piperazin-1,2-yl, morpholin-4,5-yl and thiomorpholin-4,5-yl;
more preferred R⁵ taken together with an adjacent R⁵ as well as their carbon and nitrogen of attachment is one of pyrrolidin-1,2-yl and piperidin-1,2-yl;
and combinations of the foregoing substituent assignments. It is understood that some compounds referred to herein are chiral and/or have geometric isomeric centers, for example E- and Z-isomers. The present invention encompasses all such optical, including stereoisomers and racemic mixtures, diastereomers, and geometric isomers that possess the activity that characterizes the compounds of this invention. In addition, certain compounds referred to herein can exist in solvated as well as unsolvated forms. It is understood that this invention encompasses all such solvated and unsolvated forms that possess the activity that characterizes the compounds of this invention. Compounds according to the present invention that have been modified to be detectable by some analytic technique are also within the scope of this invention. An example of such compounds is an isotopically labeled compound, such as an ¹⁸F isotopically labeled compound that may be used as a probe in detection and/or imaging techniques, such as positron emission tomography (PET) and single-photon emission computed tomography (SPECT). Another example of such compounds is an isotopically labeled compound, such as a deuterium and/or tritium labeled compound that may be used in reaction kinetic studies.

It is understood that substitutions and combinations of substitutions recited herein, whether stated explicitly or not, refer to substitutions that are consistent with the valency of the member being substituted. For example, a substitution applied to a carbon member refers to the tetravalency of C; it refers to the trivalency of N when applied to a nitrogen member; and it refers to the tetravalency of a nitrogen member that is conventionally characterized with a positive electric charge. Valence allowed options are part of the ordinary skill in the art.

The “pharmaceutically acceptable salts or esters thereof” refer to those salts, and ester forms of the compounds of the present invention that would be apparent to the pharmaceutical chemist, i.e., those that are non-toxic and that would favorably affect the pharmacological properties of said compounds of the present invention. Those compounds having favorable pharmacological properties would be apparent to the pharmaceutical chemist, i.e., those that are non-toxic and that possess such pharmacological properties to provide sufficient palatability, absorption, distribution, metabolism and excretion. Other factors, more practical in nature, that are also important in the selection are cost of raw materials, ease of crystallization, yield, stability, hygroscopicity, and flowability of the resulting bulk drug.

Representative acids and bases that may be used in the preparation of pharmaceutically acceptable salts include the following:
acids including acetic acid, 2,2-dichlorolactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid; and
bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidi ne, secondary amine, sodium hydroxide, triethanolamine, tromethamine and zinc hydroxide.

See, for example, S. M. Berge, et al., “Pharmaceutical Salts”, J. Pharm. Sci. 1977, 66:1-19, which is incorporated herein by reference. Examples of suitable esters include C₁₋₇alkyl, C₅₋₇cycloalkyl, phenyl, substituted phenyl, and phenylC₁₋₆alkyl-esters. Preferred esters include methyl esters:

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound that may not be specifically disclosed, but that converts to the specified compound in vivo after administration to the patient. Analogously, the term “compound”, when applied to compounds within the scope of this invention, shall encompass a specific compound of formula (I) or a compound (or prodrug) that converts to the specifically disclosed compound in vivo after administration, even if such prodrug is not explicitly disclosed herein. Conventional procedures for the selection and preparation of suitable prodrug derivatives are described, for example, in “Design of Prodrugs”, ed. H. Bundgaard, Elsevier, 1985.

Compounds of formula (I) comprise compounds that satisfy any one of the combinations of definitions given herein and equivalents thereof.

Embodiments of Formula I were made as described in Examples 1-23 and are selected from the group consisting of:

| EX | Compound |
|---|---|
| 1 | 8-Methyl-3-(4-methyl-piperazin-1-yl)-1H-quinoxalin-2-one; |
| 2 | 8-Methyl-3-piperazin-1-yl-1H-quinoxalin-2-one; |
| 3 | 8-Nitro-3-piperazin-1-yl-1H-quinoxalin-2-one; |
| 4 | 7,8-Difluoro-3-piperazin-1-yl-1H-quinoxalin-2-one; |
| 5 | 8-Methyl-3-(3-methyl-piperazin-1-yl)-1H-quinoxalin-2-one; |
| 6 | 3-(3-Methyl-piperazin-1-yl)-1H-quinoxalin-2-one; |
| 7 | 3-[4-(2-Hydroxy-ethyl)-piperazin-1-yl]-8-methyl-1H- |
| | quinoxalin-2-one; |
| 9 | 6-Chloro-3-(4-methyl-piperazin-1-yl)-1H-quinoxalin-2-one; |
| 10 | 7-Chloro-3-(4-methyl-piperazin-1-yl)-1H-quinoxalin-2-one; |
| 12 | 3-(4-Methyl-piperazin-1-yl)-6-trifluoromethyl-1H-quinoxalin-2-one; |
| 13 | 3-(4-Methyl-piperazin-1-yl)-7-trifluoromethyl-1H-quinoxalin-2-one; |
| 14 | 6,7-Dichloro-3-(4-methyl-piperazin-1-yl)-1H-quinoxalin-2-one; |
| 15 | 6,7-Dichloro-3-piperazin-1-yl-1H-quinoxalin-2-one; |
| 16 | 6,7-Dichloro-3-(4-methyl-[1,4]diazepan-1-yl)-1H-quinoxalin-2-one; |
| 17 | 6,7-Difluoro-3-(4-methyl-piperazin-1-yl)-1H-quinoxalin-2-one; |
| 19 | 7-Chloro-6-methyl-3-(4-methyl-piperazin-1-yl)-1H-quinoxalin-2-one; |
| 20 | 6-Chloro-7-methyl-3-(4-methyl-piperazin-1-yl)-1H-quinoxalin-2-one; |
| 21 | 6-Fluoro-3-(4-methyl-piperazin-1-yl)-1H-quinoxalin-2-one; |
| 22 | 7,8-Difluoro-3-(4-methyl-piperazin-1-yl)-1H-quinoxalin-2-one; and |
| 23 | 8-Chloro-3-(4-methyl-piperazin-1-yl)-6-trifluoromethyl-1H- |
| | quinoxalin-2-one. |

Additional embodiments of Formula I were made as components of 1:1 mixtures of regioisomers according to the synthetic methods outlined in Schemes 1 and 2 and Examples 24-28 and are selected from the group consisting of:

| EX | Compound |
|---|---|
| 24 | 3-Piperazin-1-yl-6-trifluoromethyl-1H-quinoxalin-2-one; |
| 24 | 3-Piperazin-1-yl-7-trifluoromethyl-1H-quinoxalin-2-one; |
| 25 | 6-Chloro-7-fluoro-3-(4-methyl-piperazin-1-yl)-1H-quinoxalin-2-one; |
| 25 | 7-Chloro-6-fluoro-3-(4-methyl-piperazin-1-yl)-1H-quinoxalin-2-one; |
| 26 | 7-Chloro-3-piperazin-1-yl-1H-quinoxalin-2-one; |
| 26 | 6-Chloro-3-piperazin-1-yl-1H-quinoxalin-2-one; |
| 27 | 6-Chloro-3-(3-methyl-piperazin-1-yl)-1H-quinoxalin-2-one; |
| 27 | 7-Chloro-3-(3-methyl-piperazin-1-yl)-1H-quinoxalin-2-one; |
| 28 | 3-(3-Methyl-piperazin-1-yl)-6-trifluoromethyl-1H-quinoxalin-2-one; |
| | and |
| 28 | 3-(3-Methyl-piperazin-1-yl)-7-trifluoromethyl-1H-quinoxalin-2-one. |

Embodiments of methods for making a quinoxaline compound, such as a compound of formula (I), or an enantiomer, diastereomer, racemate thereof, or a pharmaceutically acceptable salt or ester thereof, that comprise reacting a diamino compound of formula (III) with an ester of formula (IV), as indicated above, include methods wherein at least one of the following is satisfied:

R¹⁻⁷, B, and Y have any of the meanings defined hereinabove and equivalents thereof;
R is one of methyl and ethyl;
said reacting is carried out at a temperature of at least about 40° C., and in some embodiments at a temperature of about 100° C.;
said reacting is performed in a solvent whose boiling point is at least about 100° C.;
said reacting is preferably performed in toluene;
said reacting further comprises incorporating into the reaction medium a Lewis acid catalyst or a protic acid catalyst. Lanthanide triflates are examples of Lewis acid catalysts. In some specific embodiments said Lewis acid catalyst is is one of ytterbium triflate, scandium triflate, zinc chloride, copper triflate, or mixtures thereof; in some more specific embodiments said Lewis acid catalyst is ytterbium triflate; and in some specific embodiments said protic acid catalyst is p-toluenesulfonic acid, which is preferably used under Dean-Stark conditions;
the method further comprises an addition-elimination reaction of a secondary amine of formula (VI) with a compound of formula (V) that is formed in said reacting of said diamino compound with said ester, wherein in some embodiments R is chosen so that the group OR in compound of formula (VI) is a suitable leaving group in said addition-elimination reaction, and wherein in some embodiments said secondary amine is a piperazine derivative or a homopiperazine derivative;
said addition-elimination reaction is performed at a temperature of at least about 40° C., in some specific embodiments at a temperature of at least about 100° C., and in some more specific embodiments at a temperature of about 175° C.; and
performing said addition-elimination reaction further comprises incorporating into the reaction medium of a catalyst, and in some more specific embodiments said catalyst is hydroxypyridine.

Embodiments of pharmaceutical compositions for treating or preventing an H₄ receptor-mediated condition in a subject that comprise a therapeutically effective amount of at least one of an H₄ receptor modulator of formula (I), an enantiomer, diastereomer, racemate thereof, and pharmaceutically acceptable salt and ester thereof, further comprise a pharmaceutically acceptable carrier.

Embodiments of pharmaceutical compositions for inhibiting leukocyte recruitment in a subject that comprise a therapeutically effective amount of at least one of a leukocyte recruitment inhibitor of formula (I), an enantiomer, diastereomer, racemate thereof, and pharmaceutically acceptable salt and ester thereof, further comprise a pharmaceutically acceptable carrier.

Embodiments of anti-inflammatory compositions that comprise a therapeutically effective amount of at least one of anti-inflammatory compound of formula (I), an enantiomer, diastereomer, racemate thereof, and pharmaceutically acceptable salt and ester thereof, further comprise a pharmaceutically acceptable carrier.

Embodiments of methods for treating or preventing inflammation in a subject that comprise administering to the subject in connection with an inflammatory response a pharmaceutical composition comprising a therapeutically effective amount of at least one of an anti-inflammatory compound of formula (I), an enantiomer, diastereomer, racemate thereof, and pharmaceutically acceptable salt and ester thereof, include methods wherein said inflammatory response is a response to at least one of the conditions: inflammatory disorders, allergic disorders, dermatological disorders, autoimmune disease, lymphatic disorders, itchy skin, and immunodeficiency disorders.

Embodiments of methods for treating or preventing inflammation in a subject that comprise administering to the subject in connection with an inflammatory response a pharmaceutical composition comprising a therapeutically effective amount of at least one of an anti-inflammatory compound of formula (I), an enantiomer, diastereomer, racemate thereof, and pharmaceutically acceptable salt and ester thereof, include methods wherein said inflammatory response is a response to chemotherapy.

Embodiments of methods for treating or preventing inflammation in a subject that comprise administering to the subject in connection with an inflammatory response a pharmaceutical composition comprising a therapeutically effective amount of at least one of an anti-inflammatory compound of formula (I), an enantiomer, diastereomer, racemate thereof, and pharmaceutically acceptable salt and ester thereof, include methods wherein at least one of the following is satisfied: said inflammatory response is a response to a physical stimulus; said inflammatory response is a response to a chemical stimulus; said inflammatory response is a response to infection; said inflammatory response is a response to an invasion by a body that is foreign to said subject; said inflammatory response is a response to an immunological stimulus; said inflammatory response is a response to a non-immunological stimulus; said inflammatory response is a response to at least one of the conditions: allergy, asthma, chronic obstructed pulmonary disease (COPD), atherosclerosis, rheumatoid arthritis, multiple sclerosis, inflammatory bowel disease, and more specifically wherein said inflammatory bowel disease is at least one of Crohn's disease and ulcerative colitis, psoriasis, allergic rhinitis, scleroderma, autoimmune thyroid disease, immune-mediated diabetes mellitus, and lupus; said inflammatory response is a response to at least one of the conditions: myasthenia gravis, autoimmune neuropathy, and more specifically wherein said autoimmune neuropathy is Guillain-Barré neuropathy, autoimmune uveitis, autoimmune hemolytic anemia, pernicious anemia, autoimmune thrombocytopenia, temporal arteritis, anti-phospholipid syndrome, vasculitides, and more specifically wherein said vasculitides is Wegener's granulomatosis, Behcet's disease, dermatitis herpetiformis, pemphigus vulgaris, vitiligio, primary biliary cirrhosis, autoimmune hepatitis, autoimmune oophoritis, autoimmune orchitis, autoimmune disease of the adrenal gland, polymyositis, dermatomyositis, spondyloarthropathy, and more specifically wherein said spondyloarthropathy is ankylosing spondylitis, and Sjogren's syndrome; said inflammatory response is acute inflammation; said inflammatory response is allergic inflammation; and said inflammatory response is chronic inflammation. Administration “in connection with” an inflammatory response according to the present invention includes administration at a time that is at least one of prior to, at the onset of, and after inflammation is detected.

Embodiments of methods for modulating an H₄ receptor that comprise exposing an H₄ receptor to at least one of a compound of formula (I), an enantiomer, diastereomer, racemate thereof, and pharmaceutically acceptable salt and ester thereof, include methods wherein at least one of the following is satisfied: said at least one of a compound of formula (I), an enantiomer, diastereomer, racemate thereof, and pharmaceutically acceptable salt and ester thereof, modulates the H₄ receptor as a receptor antagonist, and said at least one of a compound of formula (I), an enantiomer, diastereomer, racemate thereof, and pharmaceutically acceptable salt and ester thereof, modulates the H₄ receptor as a receptor partial agonist.

If more than one active agent is administered, such as a compound of formula (I), the therapeutically effective amount may be a jointly effective amount.

An illustration of the invention is a pharmaceutical composition made by mixing at least one of a compound of formula (I), an enantiomer, diastereomer, racemate thereof, and a pharmaceutically acceptable carrier. Illustrating the invention is a process for making a pharmaceutical composition comprising mixing at least one of a compound of formula (I), an enantiomer, diastereomer, racemate thereof, and a pharmaceutically acceptable carrier.

Another example of the invention is the use of a composition that comprises at least one of a compound of formula (I), an enantiomer, diastereomer, racemate thereof, and pharmaceutically acceptable salt and ester thereof, in the preparation of a medication for treating any one of the conditions referred to herein; one of such conditions is inflammation. Another example of the invention is the use of a composition that comprises at least one of a compound of formula (I), an enantiomer, diastereomer, racemate thereof, and pharmaceutically acceptable salt and ester thereof, in the treatment or prevention of any one of the conditions referred to herein; one of such conditions is inflammation.

Compounds according to the present invention may be made according to processes within the skill of the art and/or according to processes of this invention, such as those described in the schemes and examples that follow and by matrix or combinatorial methods. To obtain the various compounds herein, starting materials may be employed that carry the ultimately desired substituents though the reaction scheme with or without protection as appropriate. Starting materials may be obtained from commercial sources or synthesized by methods known to one skilled in the art. Alternatively, it may be necessary to employ, in the place of the ultimately desired substituent, a suitable group, which may be carried through the reaction scheme and replaced as appropriate with the desired substituent. Any product containing a chiral center may be separated into its enantiomers by conventional techniques. Those of ordinary skill in the art will be able to modify and adapt the guidance provided herein to make compounds according to the present invention.

Embodiments of processes illustrated herein include, when chemically meaningful, one or more steps such as hydrolysis, halogenation, protection, and deprotection. These steps can be implemented in light of the teachings provided herein and the ordinary skill in the art.

During any of the processes for preparation of the compounds of the present invention, it may be necessary and/or desirable to protect sensitive or reactive groups on any of the molecules concerned. In addition, compounds of the invention may be modified by using protecting groups; such compounds, precursors, or prodrugs are also within the scope of the invention. This may be achieved by means of conventional protecting groups, such as those described in “Protective Groups in Organic Chemistry”, ed. J. F. W. McOmie, Plenum Press, 1973; and T. W. Greene & P. G. M. Wuts, “Protective Groups in Organic Synthesis”, 3rd ed., John Wiley & Sons, 1999. The protecting groups may be removed at a convenient subsequent stage using methods known from the art.

Compounds of formula (I) will herein be referred to as “quinoxaline compounds” for the purpose of describing methods of making such compounds. According to this terminology, “quinoxaline compounds” refer to compounds of formula (I) where B is any one of N and CR⁷, and also to compounds from which compounds of formula (I) can be formed as described herein and that have the quinoxaline framework with B being any one of N and CR⁷. Accordingly, compounds (V), (VIII) and (IX) in the reaction schemes given herein below are also referred to as quinoxaline compounds.

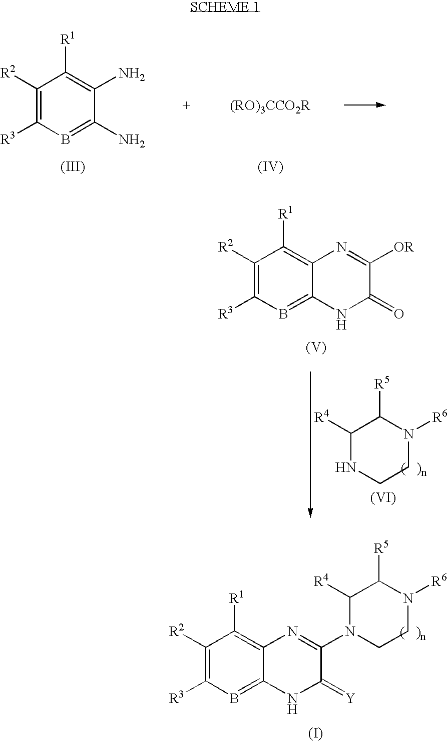

Referring to Scheme 1, there are disclosed the following notes and additions. The starting materials, including diamino compound (III), ester (IV), and secondary amine (VI) are commercially available or their syntheses are within the skill of the art.

Groups R in ester (IV) may be any of a number of C₁₋₆ alkyl groups and benzyl groups. Preferably, these groups are embodied by the same substituent, which is chosen so that the group —OR in compound (V) is a suitable leaving group in the addition-elimination reaction of compound (V) with secondary amine (VI). Preferably, R is methyl or ethyl.

Secondary amine (VI) is shown in Scheme 1 as a piperazine or homopiperazine derivative. When Y in Scheme 1 is S or NH, compound of formula (I) can be obtained by supplementing the steps in Scheme 1 with the halogenation to form compound (IX) and subsequent transformation to form compound of formula (I) according to Scheme 2 described hereinbelow.

The reaction of a diamino compound, such as compound of formula (III), with an ester, such as ester (IV), is preferably carried out at a temperature of at least about 40° C., more preferably about 100° C. Accordingly, the reaction medium for such reactions is preferably provided by a high-boiling solvent or a high-boiling mixture of solvents. Examples of such solvent media are toluene, dioxane, xylenes, 1,2-dichloroethane, and mixtures thereof. A preferred solvent is toluene. The same reaction is preferably performed with a Lewis acid catalyst, for example ytterbium triflate (Yb(OTf)₃), scandium triflate (Sc(OTf)₃, ZnCl₂, and Cu(OTf)₂. Preferably, this Lewis acid catalyst is ytterbium triflate. Alternatively, the reaction is performed with a protic acid catalyst, for example p-toluenesulfonic acid, preferably using a Dean Stark trap.

Quinoxaline compound (V) undergoes an addition-elimination reaction with a secondary amine, such as compound (VI) to form a compound of formula (I). Preferably, this reaction is performed in a solvent or solvent mixture that is suitable for such type of reaction. Examples of such solvents are toluene, dioxane, THF, benzotrifluoride, DMF, 1,2-dichloroethane, and mixtures thereof. Furthermore, this reaction is preferably performed at a temperature of at least about 40° C., more preferably at a temperature of at least about 100° C. Accordingly, the solvent for such reaction is preferably a high-boiling solvent or a high-boiling mixture of solvents. A preferred solvent medium is toluene. In other embodiments, the reaction medium contains a catalyst, such as a hydroxypyridine compound.

Reaction times are reduced as the reaction medium is heated at higher temperature and/or a catalyst is incorporated in the reaction medium. Embodiments of this invention were heated to temperatures of up to about 175° C.

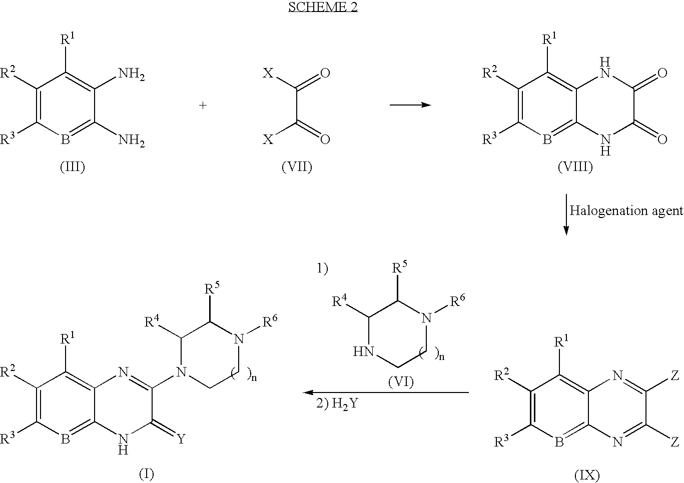

Referring to Scheme 2, there are disclosed the following notes and additions. The starting materials are commercially available or their synthesis is within the skill of the art.

Quinoxalin-dione compound (VIII) is obtained in a condensation reaction of an oxalate derivative (VII) with a suitably substituted diamino compound (III). Oxalate (VII) is preferably dimethyl oxalate, diethyl oxalate, or oxalyl chloride. Preferably, this reaction is performed at temperatures between about −20° C. and about 100° C.

Quinoxalin-dione compound (VIII) is halogenated to form quinoxaline compound (IX) where Z represents halo, preferably chloro. Thionyl chloride, thionyl bromide, and phosphorous oxychloride are examples of halogenation agents that can be used in this halogenation, which is performed under conditions known in the art.

Halogenated quinoxaline (IX) is allowed to react with suitably substituted piperazine or homopiperazine (VI) under known reaction conditions and subsequently further treated with H₂Y, wherein Y is defined above, to form a compound of formula (I).

As will be appreciated by one of ordinary skill in the art, methods according to Schemes 1 and 2 can be used to prepare a compound of formula (I) in a single isomeric form or a compound of formula (I) in the form of a regioisomeric mixture. Example 8 herein below provides, inter alia, an illustration of the implementation of the methods described herein to the production of compounds of formula (I) in the form of a regioisomeric mixture. Example 1 herein below provides, inter alia, an illustration of the implementation of the methods described herein to the production of compounds of formula (I) in a single isomeric form.

Where the processes for the preparation of the compounds according to the invention give rise to mixture of stereoisomers, these isomers may be separated by conventional techniques such as resolution, for example by formation of diastereomeric salts, kinetic resolution including variants thereof, such as dynamic resolution, preferential crystallization, biotransformation, enzymatic transformation, and preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be separated using a chiral HPLC column.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The expression of the H₄ receptor in immune cells, including some leukocytes and mast cells, establishes it as an important target for therapeutic intervention in a range of immunological and inflammatory disorders (such as allergic, chronic, or acute inflammation). Specifically H₄ receptor ligands are expected to be useful for the treatment or prevention of various mammalian disease states.

Thus, according to the invention, the disclosed compounds, whether partial agonists or antagonists of the H₄ receptor, and compositions are useful for the amelioration of symptoms associated with, the treatment of, and the prevention of, the following conditions and diseases: inflammatory disorders, allergic disorders, dermatological disorders, autoimmune disease, lymphatic disorders, and immunodeficiency disorders, including the more specific conditions and diseases given above. The disclosed compounds may also be useful as adjuvants in chemotherapy or in the treatment of itchy skin.

Aspects of the invention include (a) a pharmaceutical composition comprising at least one of a compound of formula (I), an enantiomer, diastereomer, racemate thereof, and pharmaceutically acceptable salt and ester thereof, and a preferred compound as described herein, and a pharmaceutically acceptable carrier; (b) a packaged drug comprising (1) a pharmaceutical composition comprising at least one of a compound of formula (I), an enantiomer, diastereomer, racemate thereof, and pharmaceutically acceptable salt and ester thereof, or one or more preferred compounds as described herein, and a pharmaceutically acceptable carrier, and (2) instructions for the administration of said composition for the treatment or prevention of any one of the conditions referred to herein, such as an H₄-mediated disease or condition, and more particularly inflammation.

Embodiments of this invention provide methods for treating or preventing an H₄-mediated condition in a patient, said methods comprising administering to the patient a pharmaceutically effective amount of a composition comprising at least one of a compound of formula (I), an enantiomer, diastereomer, racemate thereof, and pharmaceutically acceptable salt and ester thereof, and other disclosed or preferred compounds. In these conditions, the action of the H₄ receptor is involved. For example, the invention features a method for treating an H₄ mediated condition in a patient, said method comprising administering to the patient a pharmaceutically effective H₄-antagonizing amount of a composition comprising at least one of a compound of formula (I), an enantiomer, diastereomer, racemate thereof, and pharmaceutically acceptable salt and ester thereof. As used herein, “treating” a disorder, and grammatically related terms, mean eliminating or otherwise ameliorating the cause and/or effects thereof. Terms such as to “inhibit”, and grammatically related terms, the onset of a disorder or event, and to “prevent” a disorder or condition, and grammatically related terms, mean preventing, delaying or reducing the likelihood of such onset.

The effect of an antagonist may also be produced by an inverse agonist. Inverse agonism describes the property of a compound to actively turn off a receptor that displays constitutive activity. Constitutive activity can be identified in cells that have been forced to over-express the human H₄ receptor. Constitutive activity can be measured by examining cAMP levels or by measuring a reporter gene sensitive to cAMP levels after a treatment with a cAMP-stimulating agent such as forskolin. Cells that over-express H₄ receptors will display lower cAMP levels after forskolin treatment than non-expressing cells. Compounds that behave as H₄ agonists will dose-dependently lower forskolin-stimulated cAMP levels in H₄-expressing cells. Compounds that behave as H₄ inverse agonists will dose-dependently stimulate cAMP levels in H₄-expressing cells. Compounds that behave as H₄ antagonists will block either H₄ agonist-induced inhibition of cAMP or H₄ inverse agonist-induced increases in cAMP.

Further embodiments of the invention include disclosed compounds that are inhibitors of a mammalian histamine H₄ receptor function, inhibitors of inflammation or inflammatory responses in vivo or in vitro, modulators of the expression of a mammalian histamine H₄ receptor protein, inhibitors of polymorphonuclear leukocyte activation in vivo or in vitro, or combinations of the above, and corresponding methods of treatment, prophylaxis, and diagnosis comprising the use of a disclosed compound.

The terms “unit dose” and their grammatical equivalent forms are used herein to refer to physically discrete units suitable as unitary dosages for human patients and other animals, each unit containing a predetermined effective, pharmacologic amount of the active ingredient calculated to produce the desired pharmacological effect. The specifications for the novel unit dosage forms of this invention are determined by, and are directly dependent on, the characteristics of the active ingredient, and on the limitations inherent in the art of compounding such an active ingredient for therapeutic use in humans and other animals.

The pharmaceutical compositions can be prepared using conventional pharmaceutical excipients and compounding techniques. Examples of suitable unit dosage forms are tablets, capsules, pills, powders, powder packets, granules, wafers, and the like, segregated multiples of any unit dosage form, as well as liquid solutions, and suspensions. Some liquid forms are aqueous, whereas other embodiments of liquid forms are non-aqueous. Oral dosage forms may be elixirs, syrups, capsules, tablets and the like. Examples of solid carriers include those materials usually employed in the manufacture of pills or tablets, such as lactose, starch, glucose, methylcellulose, magnesium stearate, dicalcium phosphate, mannitol and the like, thickeners such as tragacanth and methylcellulose USP, finely divided SiO₂, polyvinylpyrrolidone, magnesium stearate, and the like. Typical liquid oral excipients include ethanol, glycerol, water and the like. All excipients may be mixed as needed with diluents (for example, sodium and calcium carbonates, sodium and calcium phosphates, and lactose), disintegrants (for example, cornstarch and alginic acid), granulating agents, lubricants (for example, magnesium stearate, stearic acid, and talc), binders (for example, starch and gelatin), thickeners (for example, paraffin, waxes, and petrolatum), flavoring agents, coloring agents, preservatives, and the like by conventional techniques known to those of ordinary skill in the art of preparing dosage forms. Coatings can be present and include, for example, glyceryl monostearate and/or glyceryl diestearate. Capsules for oral use include hard gelatin capsules in which the active ingredient is mixed with a solid diluent, and soft gelatin capsules, in which the active ingredient is mixed with water or an oil, such as peanut oil, liquid paraffin, or olive oil.

Parenteral dosage forms may be prepared using water or another sterile carrier. Parenteral solutions can be packaged in containers adapted for subdivision into individual doses. For intramuscular, intraperitoneal, subcutaneous, and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions may include suspending agents such as cellulose derivatives, sodium alginate, polyvinyl-pyrrolidone, and gum tragacanth, and a wetting agent, such as lecithin. Suitable preservatives for aqueous suspensions include ethyl and n-propyl p-hydroxybenzoate. Parenteral formulations include pharmaceutically acceptable aqueous or non-aqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Carriers for solid dosage forms include (a) fillers or extenders, (b) binders, (c) humectants, (d) disintegrating agents, (e) solution retarders, (f) absorption accelerators, (g) adsorbants, (h) lubricants, (i) buffering agents, and (j) propellants.

Compositions may also contain adjuvants such as preserving, wetting, emulsifying, and dispensing agents; antimicrobial agents such as parabens, chlorobutanol, phenol, and sorbic acid; isotonic agents such as a sugar or sodium chloride; absorption-prolonging agents such as aluminum monostearate and gelatin; and absorption-enhancing agents.

Physiologically acceptable carriers are well known in the art. Examples of liquid carriers are solutions in which compounds according to the present invention form solutions, emulsions, and dispersions. Compatible antioxidants, such as methylparaben and propylparaben, can be present in solid and/or liquid compositions, as can sweeteners.

Pharmaceutical compositions according to the present invention may include suitable emulsifiers typically used in emulsion compositions. Such emulsifiers are described in standard publications such as H. P. Fiedler, 1989, Lexikon der Hilfsstoffe für Pharmazie, Kosmetic und agrenzende Gebiete, Cantor ed., Aulendorf, Germany, and in Handbook of Pharmaceutical Excipients, 1986, American Pharmaceutical Association, Washington, D.C., and the Pharmaceutical Society of Great Britain, London, UK, which are incorporated herein by reference. Gelling agents may also be added to compositions according to this invention. Polyacrylic acid derivatives, such as carbomers, are examples of gelling agents, and more particularly, various types of carbopol, which are typically used in amounts from about 0.2% to about 2%. Suspensions may be prepared as a cream, an ointment, including a water-free ointment, a water-in-oil emulsion, an oil-in-water emulsion, an emulsion gel, or a gel.

It is anticipated that the compounds of the invention can be administered by oral or parenteral routes, including intravenous, intramuscular, intraperitoneal, subcutaneous, rectal, intracisternal, intravaginal, intravesical, topical or local administration, and by inhalation (bucal or nasal, preferably in the form of a spray). For oral administration, the compounds of the invention will generally be provided in the form of tablets, capsules, or as a solution or suspension. Other methods of administration include controlled release formulations, such as subcutaneous implants and dermal patches.

Effective doses of the compounds of the present invention may be ascertained by conventional methods. The specific dosage level required for any particular patient will depend on a number of factors, including severity of the condition, type of symptoms needing treatment, the route of administration, the weight, age, and general condition of the patient, and the administration of other medicaments.

In general, it is anticipated that the daily dose (whether administered as a single dose or as divided doses) will be in the range from about 0.01 mg to about 1000 mg per day, more usually from about 1 mg to about 500 mg per day, and most usually form about 10 mg to about 200 mg per day. Expressed as dosage per unit body weight, a typical dose will be expected to be between about 0.0001 mg/kg and about 15 mg/kg, especially between about 0.01 mg/kg and about 7 mg/kg, and most especially between about 0.15 mg/kg and 2.5 mg/kg.

Anticipated oral dose ranges include from about 0.01 to 500 mg/kg, daily, more preferably from about 0.05 to about 100 mg/kg, taken in 1-4 separate doses. Some compounds of the invention may be orally dosed in the range of about 0.05 to about 50 mg/kg daily, while others may be dosed at 0.05 to about 20 mg/kg daily. Infusion doses can range from about 1.0 to about 1.0×10⁴ μg/(kg.min) of inhibitor, admixed with a pharmaceutical carrier over a period ranging from several minutes to several days. For topical administration, compounds of the present invention may be mixed with a pharmaceutical carrier at a concentration from about 0.1 to about 10% of drug to vehicle. Capsules, tablets or other formulations (such as liquids and film-coated tablets) may be of between 0.5 and 200 mg, such as 1, 3, 5, 10, 15, 25, 35, 50 mg, 60 mg, and 100 mg and can be administered according to the disclosed methods. Daily dosages are envisaged to be, for example, between 10 mg and 5000 mg for an adult human being of normal weight.

### EXAMPLES

General Experimental:

NMR spectra were obtained on either a Bruker model DPX400 (400 MHz) or DPX500 (500 MHz) spectrometer. The format of the ¹H NMR data below is: chemical shift in ppm down field of the tetramethylsilane reference (multiplicity, coupling constant J in Hz, integration).

Mass spectra were obtained on an Agilent series 1100 MSD using electrospray ionization (ESI) in either positive or negative mode as indicated. The “mass calculated” for a molecular formula is the monoisotopic mass of the compound.

### Example 1

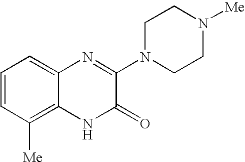 8-Methyl-3-(4-methyl-piperazin-1-yl)-1H-quinoxalin-2-one.
Method A
General Procedure 1:

A. 3-Methoxy-8-methyl-1H-quinoxalin-2-one. A mixture of 2,3-diaminotoluene (2.00 g, 16.4 mmol), trimethoxy-acetic acid methyl ester (5.37 g, 37.7 mmol), and ytterbium triflate (1.0 g, 1.64 mmol) in toluene (50 mL) was heated at 100° C. for 14 h in a sealed tube. The reaction mixture was cooled, and the precipitate was collected by vacuum filtration. After washing with toluene (2×50 mL), the precipitate was dried in vacuo to afford 1.5 g (48%) of 3-methoxy-8-methyl-1H-quinoxalin-2-one, which was used without further purification. MS (electrospray): mass calculated for C₁₀H₁₀N₂O₂, 190.2; m/z found, 191.1 [M+H]⁺. ¹H NMR (400 MHz, CDCl₃): 11.34 (br s, 1H), 7.25 (d, J=7.8 Hz, 1H), 7.13-7.10 (m, 2H), 4.14 (s, 3H), 2.59 (s, 3H).

General Procedure 2:

B. 8-Methyl-3-(4-methyl-piperazin-1-yl)-1H-quinoxalin-2-one. To a sealed tube containing 3-methoxy-8-methyl-1H-quinoxalin-2-one (50 mg, 0.26 mmol) in toluene (2 mL), was added N-methylpiperazine (0.88 mL, 0.71 mmol) and 2-hydroxypyridine (−5 mg). The mixture was heated at 120° C. for 12 h. The solution was cooled, and solvent was removed in vacuo. The crude residue was purified by silica gel chromatography (0-10% MeOH/dichloromethane (DCM)) to afford 43 mg (59%) of the title compound. MS (electrospray): mass calculated for C₁₄H₁₈N₄O, 258.3; m/z found, 259.2 [M+H]⁺. ¹H NMR (400 MHz, CDCl₃): 10.42 (br s, 1H), 7.38 (d, J=7.8 Hz, 1H), 7.15-7.12 (m, 1H), 7.05 (d, J=7.3 Hz, 1H), 4.07-4.00 (m, 4H), 2.60-2.57 (m, 4H), 2.49 (s, 3H), 2.36 (s, 3H). ¹³C NMR (400 MHz, CDCl₃): 153.8, 151.1, 133.5, 127.6, 126.9, 124.4, 124.1, 123.0, 55.6, 47.0, 47.0, 16.9.

Method B

General Procedure 3:

8-Methyl-3-(4-methyl-piperazin-1-yl)-1H-quinoxalin-2-one. A mixture of 3-methoxy-8-methyl-1H-quinoxalin-2-one (50 mg, 0.26 mmol), N-methylpiperazine (0.88 mL, 0.71 mmol) and catalytic 2-hydroxypyridine in toluene (2 mL) was heated under microwave irradiation using EmryS™ Synthesizer (Personal Chemistry) for 10 min at 170° C. Solvent was removed in vacuo, and the crude residue was purified by silica gel chromatography (0-10% MeOH/DCM) to afford 50 mg (68%) of the title compound. MS (electrospray): mass calculated for C₁₄H₁₈N₄O, 258.3; m/z found, 259.2 [M+H]⁺. ¹H NMR (400 MHz, CDCl₃): 10.42 (br s, 1H), 7.38 (d, J=7.8 Hz, 1H), 7.15-7.12 (m, 1H), 7.05 (d, J=7.3 Hz, 1H), 4.07-4.00 (m, 4H), 2.60-2.57 (m, 4H), 2.49 (s, 3H), 2.36 (s, 3H). ¹³C NMR (400 MHz, CDCl₃): 153.8, 151.1, 133.5, 127.6, 126.9, 124.4, 124.1, 123.0, 55.6, 47.0, 47.0, 16.9.

### Example 2

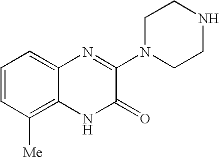 8-Methyl-3-piperazin-1-yl-1H-quinoxalin-2-one.

The reaction was carried out as described in General Procedure 2 using 3-methoxy-8-methyl-1H-quinoxalin-2-one (50 mg, 0.26 mmol) and piperazine (113 mg, 1.32 mmol). Purification afforded 23 mg (46%) of the title compound. MS (electrospray): mass calculated for C₁₃H₁₆N₄O, 244.3; m/z found, 245.2 [M+H]⁺. ¹H NMR (400 MHz, CDCl₃): 9.5 (br s, 1H), 7.39 (d, J=8.3 Hz, 1H), 7.15-7.12 (m, 1H), 7.05 (d, J=7.1 Hz, 1H), 3.98-3.95 (m, 4H), 3.08-3.02 (m, 4H), 2.42 (s, 3H).

### Example 3

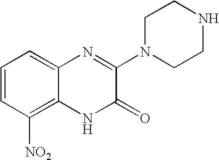 8-Nitro-3-piperazin-1-yl-1H-quinoxalin-2-one.

A. 3-Methoxy-8-nitro-1H-quinoxalin-2-one. The reaction was carried out as described in General Procedure 1 using 3-nitro-1,2-phenylenediamine (2.0 g, 13.1 mmol). After cooling to room temperature, the reaction mixture was concentrated in vacuo and used without further purification. MS (electrospray): mass calculated for C₉H₇N₃O₄, 221.0; m/z found, 222.1 [M+H]⁺. ¹H NMR (400 MHz, CD₃OD): 8.31 (d, J=8.0 Hz, 1H), 7.98 (d, J=8.0 Hz, 1H), 7.43 (t, J=8.0 Hz, 1H), 4.11 (s, 3H).

B. 8-Nitro-3-piperazin-1-yl-1H-quinoxalin-2-one. The reaction was carried out as described in General Procedure 2 with 3-methoxy-8-nitro-1H-quinoxalin-2-one (100 mg, 0.45 mmol) and piperazine (155 mg, 1.80 mmol). Purification by silica gel chromatography (0-5% MeOH/DCM) afforded 21 mg (17%) of the title compound. ¹H NMR (400 MHz, CDCl₃): 8.12 (d, J=8.0 Hz, 1H), 7.77 (d, J=8.0 Hz, 1H), 7.24 (t, J=8.0 Hz, 1H), 4.10-4.08 (m, 4H), 3.03-3.00 (m, 4H).

### Example 4

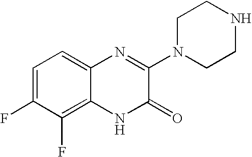 7,8-Difluoro-3-piperazin-1-yl-1H-quinoxalin-2-one.

A. 7,8-Difluoro-3-methoxy-1H-quinolin-2-one. The reaction was carried out as described in General Procedure 1 using 3,4-trifluoromethyl-1,2-phenylenediamine (680 mg, 4.68 mmol). After cooling to room temperature, the reaction mixture was concentrated in vacuo and used without further purification. MS (electrospray): mass calculated for C₁₀H₇F₂N₂O, 211.0; m/z found, 212.3 [M+H]⁺. ¹H NMR (400 MHz, CDCl₃): 12.72 (brs, 1H), 7.34-7.28 (m, 1H), 7.25-7.21 (m, 1H), 4.00 (s, 3H).

B. 7,8-Difluoro-3-(4-methyl-piperazin-1-Vl)-1H-quinoxalin-2-one. The reaction was carried out as described in General Procedure 2 with 7,8-difluoro-3-methoxy-1H-quinolin-2-one (100 mg, 0.47 mmol) and piperazine (163 mg, 1.88 mmol). Purification by silica gel chromatography (0-5% MeOH/DCM) afforded 30 mg (25%) of the title compound. ¹H NMR (400 MHz, CDCl₃): 7.23-7.19 (m, 1H), 7.02-6.95 (m, 1H), 3.96-3.94 (m, 4H), 3.04-3.00 (m, 4H).

### Example 5

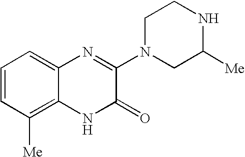 8-Methyl-3-(3-methyl-piperazin-1-yl)-1H-quinoxalin-2-one.

The reaction was carried out as described in General Procedure 2 using 3-methoxy-8-methyl-1H-quinoxalin-2-one (50 mg, 0.26 mmol) and 2-methylpiperazine (132 mg, 1.32 mmol). Purification by reversed-phase chromatography (C₁₈; 10-90% MeOH/MeCN) gave the product as a TFA salt, which was free-based using NaHCO₃ to afford 27 mg (42%) of the title compound. MS (electrospray): mass calculated for C₁₄H₁₈N₄O, 258.3; m/z found, 259.2 [M+H]⁺. ¹H NMR (400 MHz, CDCl₃): 9.5 (br s, 1H), 7.38 (d, J=8.2 Hz, 1H), 7.13-7.11 (m, 1H), 7.06 (d, J=7.1 Hz, 1H), 4.81-4.78 (m, 2H), 3.03-3.00 (m, 4H), 2.66-2.63 (m, 1H), 2.42 (s, 3H), 1.15 (d, J=6.8 Hz, 3H).

### Example 6

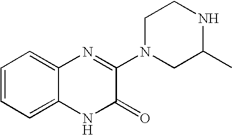 3-(3-Methyl-piperazin-1-yl)-1H-quinoxalin-2-one.

A. 3-Methoxy-1H-quinoxalin-2-one. The reaction was carried out as described in General Procedure 1 using 1,2-phenylenediamine (2.0 g, 18.5 mmol). After cooling to room temperature, the reaction mixture was concentrated in vacuo and used without further purification. MS (electrospray): mass calculated for C₉H₈N₂O₂, 176.0; m/z found, 177.1 [M+H]⁺. ¹H NMR (400 MHz, DMSO-d₆): 11.72 (br s, 1H), 7.77-7.74 (m, 1H), 7.54-7.52 (m, 1H), 7.31-7.21 (m, 2H), 3.95 (s, 3H).

B. 3-(3-Methyl-piperazin-1-yl)-1H-quinoxalin-2-one. The reaction was carried out as described in General Procedure 2 with 3-methoxy-1H-quinoxalin-2-one (100 mg, 0.56 mmol) and piperazine (226 mg, 2.22 mmol). Purification by silica gel chromatography (0-5% MeOH/DCM) afforded 50 mg (36%) of the title compound. MS (electrospray): mass calculated for C₁₃H₁₆N₄O, 244.1; m/z found, 245.2 [M+H]⁺. ¹H NMR (400 MHz, DMSO-d₆): 9.05 (brs, 1H), 7.32-7.30 (m, 1H), 7.21-7.08 (m, 3H), 4.81-4.79 (m, 2H), 3.35-3.02 (m, 4H), 2.56-2.53 (m, 1H), 1.19 (d, J=6.8 Hz, 3H).

### Example 7

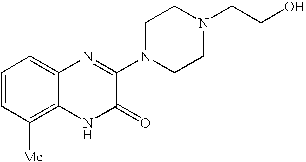

3-[4-(2-Hydroxy-ethyl)-piperazin-1-yl]-8-methyl-1H-quinoxalin-2-one. The reaction was carried out as described in General Procedure 2 using 3-methoxy-8-methyl-1H-quinoxalin-2-one (50 mg, 0.26 mmol) and N-hydroxyethylpiperazine (0.16 mL, 1.32 mmol). Purification by reversed-phase chromatography (C₁₈; 10-90% MeOH/MeCN, 1% TFA) gave the product as a TFA salt, which was free-based using NaHCO₃ to afford 10 mg (13%) of the title compound. MS (electrospray): mass calculated for C₁₅H₂₀N₄O₂, 288.3; m/z found, 289.2 [M+H]⁺. ¹H NMR (400 MHz, CDCl₃): 9.7 (br s, 1H), 7.39 (d, J=8.3 Hz, 1H), 7.14-7.10 (m, 1H), 7.06 (d, J=7.3 Hz, 1H), 4.03-4.00 (m, 4H), 3.68-3.65 (m, 2H), 2.88-2.70 (m, 6H), 2.43 (s, 3H).

### Example 8

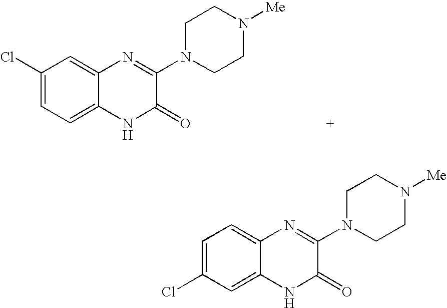 6-Chloro-3-(4-methyl-piperazin-1-yl)-1H-quinoxalin-2-one; and 7-Chloro-3-(4-methyl-piperazin-1-yl)-1H-quinoxalin-2-one.
Method A

A. 6-Chloro-3-methoxy-1H-quinoxalin-2-one and 7-Chloro-3-methoxy-1H-quinoxalin-2-one. The reaction was carried out as described in General Procedure 1 using 4-chloro-1,2-phenylenediamine (500 mg, 3.50 mmol), trimethoxy-acetic acid methyl ester (862 mg, 5.25 mmol), and ytterbium triflate (43 mg, 0.07 mmol). The precipitate was collected and dissolved in chloroform (20 mL). The solution was de-colorized with charcoal, filtered, and concentrated in vacuo to afford 1.4 g (48%) of the product as a 1:1 mixture of regioisomers. MS (electrospray): mass calculated for C₉H₇ClN₂O₂, 210.0; m/z found, 211.1 [M+H]⁺. ¹H NMR (400 MHz, CD₃OD): 7.59-7.54 (m, 2H), 7.33 (dd, J=8.8, 2.3, 1H), 7.26-7.22 (m, 3H), 4.07-4.00 (m, 6H). The above mixture was purified by silica gel chromatography (20-50% ethyl acetate/hexanes) to afford 100 mg of 6-chloro-3-methoxy-1H-quinoxalin-2-one and 150 mg of 7-chloro-3-methoxy-1H-quinoxalin-2-one. 6-Chloro-3-methoxy-1H-quinoxalin-2-one: ¹H NMR (400 MHz, CDCl₃): 11.45 (br s, 1H), 7.57 (d, J=8.6 Hz, 1H), 7.35 (d, J=2.3 Hz, 1H), 7.27-7.24 (dd, J=8.6, 2.3 Hz, 1H), 4.16-4.13 (m, 3H). 7-Chloro-3-methoxy-1H-quinoxalin-2-one: ¹H NMR (400 MHz, CDCl₃): 11.40 (br s, 1H), 7.66 (d, J=2.3 Hz, 1H), 7.34-7.32 (dd, J=8.6, 2.3 Hz, 1H), 7.27-7.25 (d, J=8.6 Hz, 1H), 4.15-4.13 (m, 3H).

B. 6-Chloro-3-(4-methyl-piperazin-1-yl)-1H-quinoxalin-2-one and 7-Chloro-3-(4-methyl-piperazin-1-yl)-1H-quinoxalin-2-one. The reaction was carried out as described in General Procedure 2 with a mixture of 6-chloro-3-methoxy-1H-quinoxalin-2-one and 7-chloro-3-methoxy-1H-quinoxalin-2-one (100 mg, 0.48 mmol total), and N-methylpiperazine (0.27 mL, 2.38 mmol). Purification by reversed-phase chromatography (C₁₈; 10-90% MeOH/MeCN, 1% TFA) gave the product as the TFA salt, which was free-based using NaHCO₃ to afford 30 mg (23%) of a 1:1 mixture of the title regioisomers. MS (electrospray): mass calculated for C₁₃H₁₅ClN₄O, 278.1; m/z found, 279.1 [M+H]⁺. ¹H NMR (400 MHz, CDCl₃): 11.12 (br s, 2H), 7.50 (d, J=2.3 Hz, 1H), 7.42 (d, J=8.6 Hz, 1H), 7.18-7.13 (m, 2H), 7.10 (d, J=2.3 Hz, 1H), 7.02 (d, J=8.6 Hz, 1H), 4.09-4.04 (m, 8H), 2.58-2.55 (m, 8H), 2.36 (s, 6H).

Method B

General Procedure 4:

A. 2,3,6-Trichloro-quinoxaline. A mixture of 6-chloro-1,4-dihydro-quinoxaline-2,3-dione (500 mg, 2.54 mmol) and phosphorous oxychloride (3 mL) was treated with DMF (−0.1 mL), and the reaction mixture was heated at reflux for 16 h. The solution was cooled to room temperature and carefully poured onto ice. The resultant solid was collected, washed with water (2×20 mL), and dried in vacuo to give 510 mg (86%) of 2,3,6-trichloro-quinoxaline, which was used without further purification. MS (electrospray): mass calculated for C₈H₃Cl₃N₂, 233.9; m/z found, 235.2 [M+H]⁺. ¹H NMR (400 MHz, CDCl₃): 8.03 (d, J=2.3 Hz, 1H), 7.98 (d, J=9.1 Hz, 1H), 7.77-7.74 (dd, J=9.1, 2.3 Hz, 2H).

General Procedure 5:

B. 2,6-Dichloro-3-(4-methyl-piperazin-1-yl)-quinoxaline and 3,6-Dichloro-2-(4-methVi-piperazin-1-yl)-quinoxaline. To a solution of 2,3,6-trichloro-quinoxaline (100 mg, 0.43 mmol) in DMF (3 mL) was added N-methylpiperazine (0.47 mL, 0.43 mmol). The reaction mixture was stirred for 12 h, and then the solvent was removed in vacuo. The residue was purified by silica gel chromatography to give 47 mg of 2,6-dichloro-3-(4-methyl-piperazin-1-yl)-quinoxaline and 28 mg of 3,6-dichloro-2-(4-methyl-piperazin-1-yl)-quinoxaline. 2,6-Dichloro-3-(4-methyl-piperazin-1-yl)-quinoxaline: ¹H NMR (400 MHz, CDCl₃): 7.80 (d, J=2.3 Hz, 1H), 7.77 (d, J=8.8 Hz, 1H), 7.46-7.43 (dd, J=8.8, 2.3 Hz, 2H), 3.63-3.62 (m, 4H), 2.64-2.61 (m, 4H), 2.38 (s, 3H). 3,6-Dichloro-2-(4-methyl-piperazin-1-yl)-quinoxaline: ¹H NMR (400 MHz, CDCl₃): 7.85 (d, J=2.3 Hz, 1H), 7.75 (d, J=8.8 Hz, 1H), 7.59-7.56 (dd, J=8.8, 2.3 Hz, 2H), 3.63-3.61 (m, 4H), 2.64-2.62 (m, 4H), 2.39 (s, 3H).

General Procedure 6:

C. 6-Chloro-3-(4-methyl-piperazin-1-yl)-1H-quinoxalin-2-one and 7-Chloro-3-(4-methyl-piperazin-1-yl)-1H-quinoxalin-2-one. A 1:1 mixture of 2,6-dichloro-3-(4-methyl-piperazin-1-yl)-quinoxaline and 3,6-dichloro-2-(4-methyl-piperazin-1-yl)-quinoxaline (50 mg, 0.17 mmol total) was dissolved in THF (2 mL), and 1 M LiOH (1 mL) was added. The solution was heated at reflux for 16 h. The reaction mixture was partitioned between water (5 mL) and chloroform (5 mL). The organic phase was dried, and solvent was evaporated to afford 20 mg (43%) of a 1:1 mixture of the title regioisomers. MS (electrospray): mass calculated for C₁₃H₁₅ClN₄O, 278.1; m/z found, 279.1 [M+H]⁺. ¹H NMR (400 MHz, CDCl₃): 11.12 (br s, 2H), 7.50 (d, J=2.3 Hz, 1H), 7.42 (d, J=8.6 Hz, 1H), 7.18-7.13 (m, 2H), 7.10 (d, J=2.3 Hz, 1H), 7.02 (d, J=8.6 Hz, 1H), 4.09-4.04 (m, 8H), 2.58-2.55 (m, 8H), 2.36 (s, 6H).

### Example 9

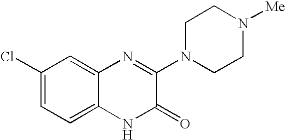 6-Chloro-3-(4-methyl-piperazin-1-yl)-1H-quinoxalin-2-one.

The reaction was carried out as described in General Procedure 6 with 2,6-dichloro-3-(4-methyl-piperazin-1-yl)-quinoxaline (63 mg, 0.21 mmol, affording the title compound in 51% yield (30 mg). MS (electrospray): mass calculated for C₁₃H₁₅ClN₄O, 278.1; m/z found, 279.1 [M+H]⁺. ¹H NMR (400 MHz, DMSO-d₆): 12.21 (br s, 1H), 7.36 (d, J=2.3 Hz, 1H), 7.20-7.17 (dd, J=8.6, 2.3 Hz, 1H), 7.13 (d, J=8.6 Hz, 1H), 3.94-3.91 (m, 4H), 2.42-2.40 (m, 4H), 2.21 (s, 3H).

### Example 10

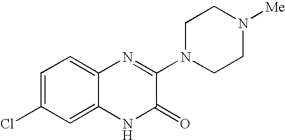 7-Chloro-3-(4-methyl-piperazin-1-yl)-1H-quinoxalin-2-one.

The reaction was carried out as described in General Procedure 6 using 3,6-dichloro-2-(4-methyl-piperazin-1-yl)-quinoxaline (20 mg, 0.07 mmol), affording the title compound in 34% yield (7 mg). MS (electrospray): mass calculated for C₁₃H₁₅ClN₄O, 278.1; m/z found, 279.1 [M+H]⁺. ¹H NMR (400 MHz, CDCl₃): 9.88 (br s, 1H), 7.42 (d, J=8.6 Hz, 1H), 7.17-7.14 (dd, J=8.6, 2.3 Hz, 1H), 7.04 (d, J=2.3 Hz, 1H), 4.04-4.02 (m, 4H), 2.58-2.55 (m, 4H), 2.35 (s, 3H).

### Example 11

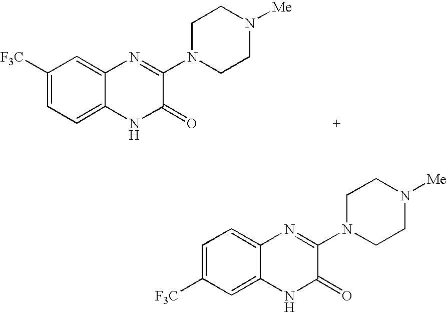 3-(4-Methyl-piperazin-1-yl)-6-trifluoromethyl-1H-qui noxalin-2-one; and 3-(4-Methyl-piperazin-1-yl)-7-trifluoromethyl-1H-quinoxalin-2-one.

A. 3-Methoxy-6-trifluoromethyl-1H-quinoxalin-2-one and 3-Methoxy-7-trifluoromethyl-1H-quinoxalin-2-one. The reaction was carried out as described in General Procedure 1 using 4-trifluoromethyl-1,2-phenylenediamine (2.00 g, 11.35 mmol). The cooled reaction mixture was concentrated in vacuo, and the crude residue was dissolved in ethyl acetate. The solution was passed through a plug of silica gel and concentrated, yielding 1.5 g (54%) of the product as a 1:1 mixture of regioisomers. MS (electrospray): mass calculated for C₁₀H₇F₃N₂O₂, 244.1; m/z found, 245.1 [M+H]⁺. ¹H NMR (400 MHz, CDCl₃): 12.21 (br s, 2H), 7.96 (d, J=1.5 Hz, 1H), 7.75 (d, J=8.3 Hz, 1H), 7.62-7.53 (m, 3H), 7.47 (d, J=8.3 Hz, 1H), 4.21-4.18 (m, 6H). A portion of this 1:1 mixture was purified by silica gel chromatography, eluting with 20-50% THF/hexanes, to give 500 mg of 3-methoxy-6-trifluoromethyl-1H-quinoxalin-2-one and 200 mg of 3-methoxy-6-trifluoromethyl-1H-quinoxalin-2-one. 3-Methoxy-6-trifluoromethyl-1H-quinoxalin-2-one: ¹H NMR (400 MHz, CDCl₃): 12.43 (br s, 1H), 7.94 (d, J=1.5 Hz, 1H), 7.60-7.58 (dd, J=8.3, 1.5 Hz, 1H), 7.49 (d, J=8.3 Hz, 1H), 4.17-4.15 (m, 3H). 3-Methoxy-6-trifluoromethyl-1H-quinoxalin-2-one: ¹H NMR (400 MHz, CDCl₃): 12.18 (br s, 1H), 7.74 (d, J=8.6 Hz, 1H), 7.66 (d, J=1.5 Hz, 1H), 7.55-7.53 (dd, J=8.6, 1.5 Hz, 1H), 4.18 (s, 3H).

B. 3-(4-Methyl-piperazin-1-yl)-6-trifluoromethyl-1H-quinoxalin-2-one and 3-(4-Methyl-piperazin-1-yl)-7-trifluoromethyl-1H-quinoxalin-2-one. The reaction was carried out as described in General Procedure 3 with a 1:1 mixture of 3-methoxy-6-trifluoromethyl-1H-quinoxalin-2-one and 3-methoxy-7-trifluoromethyl-1H-quinoxalin-2-one (96 mg, 0.37 mmol total). Purification by reversed-phase chromatography (C₁₈; 10-90% MeOH/MeCN, 1% TFA) gave the product as the TFA salt, which was free-based using NaHCO₃ to afford 60 mg (56%) of a 1:1 mixture of the title regioisomers. MS (electrospray): mass calculated for C₁₄H₁₅F₃N₄O, 312.2; m/z found, 313.1 [M+H]⁺. ¹H NMR (400 MHz, DMSO-d₆): 12.35 (br s, 2H), 7.61 (d, J=1.5 Hz, 1H), 7.50-7.40 (m, 4H), 7.30 (d, J=8.3 Hz, 1H), 4.00-3.95 (m, 8H), 2.43-2.40 (m, 8H), 2.20 (s, 3H).

### Example 12

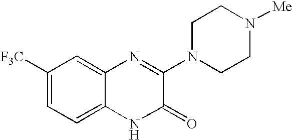 3-(4-Methyl-piperazin-1-yl)-6-trifluoromethyl-1H-quinoxalin-2-one.

The reaction was carried out as described in General Procedure 3 with 3-methoxy-6-trifluoromethyl-1H-quinoxalin-2-one (100 mg, 0.41 mmol). Purification by reversed-phase chromatography (C₁₈; 10-90% MeOH/MeCN, 1% TFA) gave the product as the TFA salt, which was free-based using NaHCO₃ to afford 70 mg (55%) of the title compound. MS (electrospray): mass calculated for C₁₄H₁₅F₃N₄O, 312.2; m/z found, 313.1 [M+H]⁺. ¹H NMR (400 MHz, CDCl₃): 11.34 (br s, 2H), 7.79 (d, J=1.5 Hz, 1H), 7.43-7.41 (dd, J=8.3, 1.5 Hz, 1H), 7.18 (d, J=8.3 Hz, 2H), 4.13-4.10 (m, 4H), 2.61-2.59 (m, 4H), 2.37 (s, 3H).

### Example 13

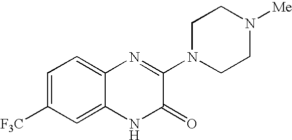 3-(4-Methyl-piperazin-1-yl)-7-trifluoromethyl-1H-quinoxalin-2-one.

The reaction was carried out as described in General Procedure 3 with 3-methoxy-7-trifluoromethyl-1H-quinoxalin-2-one (100 mg, 0.41 mmol). Purification by reversed-phase chromatography (Cl₈; 10-90% MeOH/MeCN, 1% TFA) gave the product as the TFA salt, which was free-based using NaHCO₃ to afford 70 mg (55%) of the title compound. MS (electrospray): mass calculated for C₁₄H₁₅F₃N₄O, 312.2; m/z found, 313.1 [M+H]⁺. ¹H NMR (400 MHz, CDCl₃): 10.55 (br s, 1H), 7.56 (d, J=8.3 Hz, 1H), 7.45-7.46 (dd, J=8.3, 1.5 Hz, 1H), 7.31 (d, J=1.5 Hz, 1H), 4.18-4.15 (m, 4H), 2.60-2.57 (m, 4H), 2.36 (s, 3H).

### Example 14

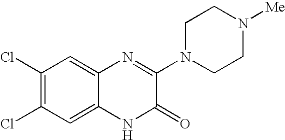 6,7-Dichloro-3-(4-methyl-piperazin-1-yl)-1H-quinoxalin-2-one.
Method A

A. 6,7-Dichloro-3-methoxy-1H-quinoxalin-2-one. The reaction was carried out as described in General Procedure 1 using 4,5-dichloro-1,2-phenylenediamine (300 mg, 1.69 mmol). The precipitate was collected by vacuum filtration and used without further purification (150 mg, 36%). MS (electrospray): mass calculated for C₉H₆Cl₂N₂O₂, 243.9; m/z found, 245.0 [M+H]⁺. ¹H NMR (400 MHz, DMSO-d₆): 7.71 (s, 1H), 7.35 (s, 1H), 3.95 (s, 3H). ¹³C NMR (400 MHz, DMSO-d₆): 156.5, 150.3, 130.7, 130.5, 128.8, 127.3, 125.2, 116.2, 54.8.

B. 6,7-Dichloro-3-(4-methyl-piperazin-1-yl)-1H-quinoxalin-2-one. The reaction was carried out as described in General Procedure 2 with 6,7-dichloro-3-methoxy-1H-quinoxalin-2-one (292 mg, 1.19 mmol). Purification by silica gel chromatography (0-5% MeOH/DCM) afforded 180 mg (49%) of the title compound. MS (electrospray): mass calculated for C₁₃H₁₄Cl₂N₄O, 312.1; m/z found, 313.1 [M+H]⁺. ¹H NMR (400 MHz, CDCl₃): 9.98 (s, 1H), 7.60 (s, 1H), 7.09 (s, 1H), 4.01-3.98 (m, 4H), 2.43-2.39 (m, 4H), 2.20 (s, 3H). ¹³C NMR (400 MHz, CDCl₃): 153.0, 151.4, 132.9, 128.5, 128.3, 127.5, 126.8, 115.6, 55.3, 46.6, 46.1, 30.0.

Method B

A. 2,6,7-Trichloro-3-(4-methyl-piperazin-1-yl)-quinoxaline. The reaction was carried out as described in General Procedure 5 with commercially available 2,3,6,7-tetrachloro-quinoxaline (1.00 g, 3.76 mmol) and N-methylpiperazine (0.43 mL, 3.95 mmol). Purification by silica gel chromatography (4% MeOH/DCM) afforded 1.1 g (89%) of 2,6,7-trichloro-3-(4-methyl-piperazin-1-yl)-quinoxaline. MS (electrospray): mass calculated for C₁₃H₁₃Cl₃N₄, 330.0; m/z found, 331.1 [M+H]⁺. ¹H NMR (400 MHz, CDCl₃): 7.95 (s, 1H), 7.93 (s, 1H), 3.65-3.62 (m, 4H), 2.65-2.62 (m, 4H), 2.38 (s, 3H).

B. 6.7-Dichloro-3-(4-methyl-piperazin-1-vI)-1H-quinoxalin-2-one. The reaction was carried out as described in General Procedure 6 with 2,6,7-trichloro-3-(4-methyl-piperazin-1-yl)-quinoxaline (100 mg, 0.30 mmol) and 3 M KOH (1 mL) to afford 60 mg (64%) of the title compound. MS (electrospray): mass calculated for C₁₃H₁₄Cl₂N₄O, 312.1; m/z found, 313.1 [M+H]⁺. ¹H NMR (400 MHz, CDCl₃): 9.98 (s, 1H), 7.60 (s, 1H), 7.09 (s, 1H), 4.02-3.99 (m, 4H), 2.43-2.40 (m, 4H), 2.20 (s, 3H). ¹³C NMR (400 MHz, CDCl₃): 153.0, 151.4, 132.9, 128.5, 128.3, 127.5, 126.8, 115.6, 55.3, 46.6, 46.1, 30.0.

### Example 15

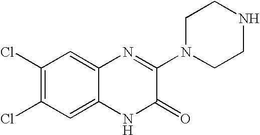 6,7-Dichloro-3-piperazin-1-yl-1H-quinoxalin-2-one.

The reaction was carried out as described in General Procedure 3 with 6,7-dichloro-3-methoxy-1H-quinoxalin-2-one (100 mg, 0.41 mmol) and piperazine (177 mg, 2.05 mmol). Purification by reversed-phase chromatography (C₁₈; 10-90% MeOH/MeCN, 1% TFA) gave the product as the TFA salt, which was free-based using NaHCO₃ to afford 25 mg (16%) of the title compound. MS (electrospray): mass calculated for C₁₂H₁₂Cl₂N₄O, 298.0; m/z found, 299.1 [M+H]⁺. ¹H NMR (400 MHz, CDCl₃): 7.58 (s, 1H), 7.15 (s, 1H), 4.05-4.02 (m, 4H), 3.06-3.03 (m, 4H). ¹³C NMR (500 MHz, CDCl₃): 153.4, 151.1, 133.3, 128.2, 128.0, 127.9, 127.0, 115.5, 48.5, 46.7, 30.1.

### Example 16

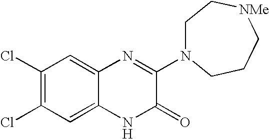 6,7-Dichloro-3-(4-methyl-[1,4]diazepan-1-yl)-1H-quinoxalin-2-one.

A. 2,6,7-Trichloro-3-(4-methyl-[1,4]diazepan-1-yl)-quinoxaline. The reaction was carried out as described in General Procedure 5 with commercially available 2,3,6,7-tetrachloro-quinoxaline (100 mg, 0.38 mmol) and N-methyl-homomethylpiperazine (0.05 mL, 0.38 mmol). Purification by silica gel chromatography (4% MeOH/DCM) afforded 25 g (19%) of 2,6,7-trichloro-3-(4-methyl-[1,4]diazepan-1-yl)-quinoxaline. ¹H NMR (400 MHz, CDCl₃): 7.90 (s, 1H), 7.83 (s, 1H), 3.92-3.83 (m, 4H), 2.87-2.85 (m, 2H), 2.68-2.65 (m, 2H), 2.41 (s, 3H), 2.13-2.10 (m, 2H).

B. 6,7-Dichloro-3-(4-methyl-[1,4]diazepan-1-yl)-1H-quinoxalin-2-one. The reaction was carried out as described in General Procedure 6 with 2,6,7-trichloro-3-(4-methyl-piperazin-1-yl)-quinoxaline (25 mg, 0.07 mmol) and 3 M KOH (1 mL) to afford 10 mg (42%) of the title compound. ¹H NMR (400 MHz, CDCl₃): 7.51 (s, 1H), 7.09 (s, 1H), 4.10-4.02 (m, 4H), 2.86-2.84 (m, 2H), 2.63-2.60 (m, 2H), 2.41 (s, 3H), 2.09-2.07 (m, 2H).

### Example 17

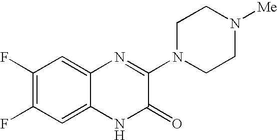 6,7-Difluoro-3-(4-methyl-piperazin-1-yl)-1H-quinoxalin-2-one.

A. 6,7-Difluoro-3-methoxy-1H-quinoxalin-2-one. The reaction was carried out as described in General Procedure 1 using 4,5-difluoro-1,2-phenylenediamine (1.00 mg, 6.90 mmol). The precipitate was collected by vacuum filtration and used without further purification (1.44 g, 98%). MS (electrospray): mass calculated for C₉H₆F₂N₂O₂, 212.2; m/z found, 213.1 [M+H]⁺. ¹H NMR (400 MHz, DMSO-d₆): 12.43 (br s, 1H), 7.63-7.58 (dd, J=11.1, 8.1 Hz, 1H), 7.15 (d, J=11.1, 8.1 Hz, 1H), 3.94 (s, 3H).

B. 6,7-Difluoro-3-(4-methyl-piperazin-1-Vl)-1H-quinoxalin-2-one. The reaction was carried out as described in General Procedure 2 with 6,7-difluoro-3-methoxy-1H-quinoxalin-2-one (200 mg, 0.94 mmol). Purification by silica gel chromatography (0-5% MeOH/DCM) afforded 180 mg (49%) of the title compound. MS (electrospray): mass calculated for C₁₃H₁₄F₂N₄O, 280.3; m/z found, 281.2 [M+H]⁺. ¹H NMR (400 MHz, CDCl₃): 7.31-7.26 (m, 1H), 7.09 (dd, J=10.6, 7.8 Hz, 1H), 4.03-4.00 (m, 4H), 2.59-2.56 (m, 4H), 2.35 (s, 3H).

### Example 18

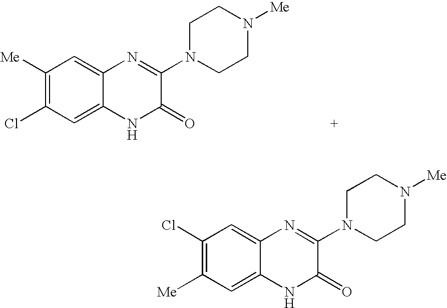 7-Chloro-6-methyl-3-(4-methyl-piperazin-1-yl)-1H-quinoxalin-2-one; and 6-Chloro-7-methyl-3-(4-methyl-piperazin-1-yl)-1H-quinoxalin-2-one.

A. 7-Chloro-3-methoxy-6-methyl-1H-quinoxalin-2-one and 6-Chloro-3-methoxy-7-methyl-1H-quinoxalin-2-one. The reaction was carried out as described in General Procedure 1 using 4-chloro-5-methyl-1,2-phenylenediamine (200 mg, 1.28 mmol). After cooling to room temperature, the solution was concentrated in vacuo and used without further purification. MS (electrospray): mass calculated for C₁₀H₉ClN₂O₂, 224.0; m/z found, 225.1 [M+H]⁺.

B. 7-Chloro-6-methyl-3-(4-methyl-piperazin-1-yl)-1H-quinoxalin-2-one and 6-Chloro-7-methyl-3-(4-methyl-piperazin-1-yl)-1H-quinoxalin-2-one. The reaction was carried out as described in General Procedure 2 with a crude mixture of 6-chloro-3-methoxy-7-methyl-1H-quinoxalin-2-one and 7-chloro-3-methoxy-6-methyl-1H-quinoxalin-2-one (1.28 mmol total). Purification by silica gel chromatography (0-5% MeOH/DCM) afforded 30 mg (8%) of a 1:1 mixture of the title regioisomers. MS (electrospray): mass calculated for C₁₄H₁₇ClN₄O, 292.1; m/z found, 293.1 [M+H]⁺. ¹H NMR (400 MHz, CDCl₃): 10.17 (br s, 2H), 7.52 (s, 1H), 7.37 (s, 1H), 7.09 (s, 1H), 6.92 (s, 1H), 4.10-4.07 (m, 4H), 2.68-2.66 (m, 4H), 2.42-2.38 (s, 6H).

### Example 19

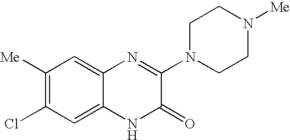 7-Chloro-6-methyl-3-(4-methyl-piperazin-1-yl)-1H-quinoxalin-2-one.

A. 2,3,6-Trichloro-7-methyl-quinoxaline. The reaction was carried out according to General Procedure 4 with 6-chloro-7-methyl-1,4-dihydro-quinoxaline-2,3-dione (1.30 g, 6.19 mmol). The resultant solid was recrystallized from ethyl acetate/hexanes to give 700 mg (46%) of 2,3,6-trichloro-7-methyl-quinoxaline. MS (electrospray): mass calculated for C₁₄H₁₆Cl₂N₄, 245.8; m/z found, 246.9 [M+H]⁺. ¹H NMR (400 MHz, CDCl₃): 8.03 (s, 1H), 7.88 (s, 1H), 2.61 (s, 3H).

B. 2,6-Dichloro-7-methyl-3-(4-methyl-piperazin-1-yl)-guinoxaline and 2,7-Dichloro-6-methyl-3-(4-methyl-piperazin-1-vi)-quinoxaline. The reaction was carried out according to General Procedure 5 with 2,3,6-trichloro-7-methyl-quinoxaline (500 mg, 2.04 mmol). Purification using silica gel chromatography (2:1 hexanes/THF) afforded 105 mg of 2,6-dichloro-7-methyl-3-(4-methyl-piperazin-1-yl)-quinoxaline and 134 mg of 2,7-dichloro-6-methyl-3-(4-methyl-piperazin-1-yl)-quinoxaline. 2,6-Dichloro-7-methyl-3-(4-methyl-piperazin-1-yl)-quinoxaline: MS (electrospray): mass calculated for C₁₄H₁₆Cl₂N₄, 310.1; m/z found, 311.2 [M+H]⁺. ¹H NMR (400 MHz, CDCl₃): 7.85 (s, 1H), 7.70 (s, 1H), 3.59-3.56 (m, 4H), 2.64-2.62 (m, 4H), 2.52 (s, 3H), 2.38 (s, 3H). 2,7-Dichloro-6-methyl-3-(4-methyl-piperazin-1-yl)-quinoxaline: MS (electrospray): mass calculated for C₁₄H₁₆Cl₂N₄, 310.1; m/z found, 311.2 [M+H]⁺. ¹H NMR (400 MHz, CDCl₃): 7.86 (s, 1H), 7.68 (s, 1H), 3.61-3.58 (m, 4H), 2.64-2.60 (m, 4H), 2.53 (s, 3H), 2.38 (s, 3H).

C. 7-Chloro-6-methyl-3-(4-methyl-piperazin-1-yl)-1H-quinoxalin-2-one. The reaction was carried out according to General Procedure 6 with 2,7-dichloro-6-methyl-3-(4-methyl-piperazin-1-yl)-quinoxaline (50 mg, 0.16 mmol). Evaporation of solvent from the organic extract yielded 10 mg (21%) of the title compound. MS (electrospray): mass calculated for C₁₄H₁₇ClN₄O, 292.1; m/z found, 293.3 [M+H]⁺. ¹H NMR (400 MHz, DMSO-d₆): 12.05 (br s, 1H), 7.35 (s, 1H), 7.15 (s, 1H), 3.90-3.87 (m, 4H), 2.43-2.42 (m, 4H), 2.31 (s, 3H), 2.20 (s, 3H).

### Example 20

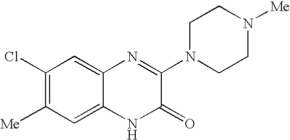 6-Chloro-7-methyl-3-(4-methyl-piperazin-1-yl)-1H-quinoxalin-2-one.

The reaction was carried out according to General Procedure 6 with 2,6-dichloro-7-methyl-3-(4-methyl-piperazin-1-yl)-quinoxaline (126 mg, 0.41 mmol). Evaporation of solvent from the organic extract yielded 85 mg (67%) of the title compound. MS (electrospray): mass calculated for C₁₄H₁₇ClN₄O, 292.1; m/z found, 293.3 [M+H]⁺. ¹H NMR (400 MHz, DMSO-d₆): 12.17 (br s, 1H), 7.38 (s, 1H), 7.06 (s, 1H), 3.90-3.87 (m, 4H), 2.42-2.40 (m, 4H), 2.32 (s, 3H), 2.20 (s, 3H).

### Example 21

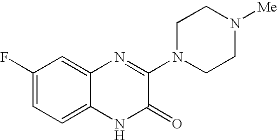 6-Fluoro-3-(4-methyl-piperazin-1-yl)-1H-quinoxalin-2-one.

A. 6-Fluoro-1.4-dihydro-quinoxaline-2,3-dione. 4-Fluoro-1,2-phenylenediamine (1.00 g, 7.92 mmol) was heated at 130° C. with diethyl oxalate (6 mL) for 16 h. The precipitate was collected by vacuum filtration and washed with hexanes (2×20 mL). This crude product was dried in air and used without further purification (1.32 g, 92%). MS (electrospray): mass calculated for C₈H₅FN₂O₂, 180.0; m/z found, 181.2 [M+H]⁺. ¹H NMR (400 MHz, CD₃OD): 7.17-7.13 (m, 1H), 6.95-6.90 (m, 2H).

B. 2,3-Dichloro-6-fluoro-quinoxaline. The reaction was carried out according to General Procedure 4 with 6-fluoro-1,4-dihydro-quinoxaline-2,3-dione (1.30 g, 7.22 mmol). The resultant solid was recrystallized from ethyl acetate/hexanes to give 500 mg (32%) of 2,3-dichloro-6-fluoro-quinoxaline. MS (electrospray): mass calculated for C₈H₃Cl₂FN₄, 216.0; m/z found, 217.1 [M+H]⁺. ¹H NMR (400 MHz, CDCl₃): 8.07-8.03 (dd, J=9.4, 5.3 Hz, 1H), 7.69-7.66 (dd, J=9.0, 2.8 Hz, 1H), 7.60-7.51 (m, 1H).

C. 2-Chloro-6-fluoro-3-(4-methyl-piperazin-1-yl)-quinoxaline. The reaction was carried out according to General Procedure 5 with 2,3-dichloro-6-fluoro-quinoxaline (240 mg, 1.11 mmol), N-methylpiperazine (0.12 mL, 1.11 mmol) in DCM (2 mL). Purification by silica gel chromatography (10-30% THF/hexanes) afforded 200 mg (65%) of the product. ¹H NMR (400 MHz, CDCl₃): 7.88-7.84 (dd, J=9.1, 5.8 Hz, 1H), 7.46-7.43 (dd, J=9.4, 2.8 Hz, 1H), 7.33-7.28 (m, 1H).

D. 6-Fluoro-3-(4-methVI-piperazin-1-yl)-1H-quinoxalin-2-one. The reaction was carried out according to General Procedure 6 with 2,6-dichloro-7-methyl-3-(4-methyl-piperazin-1-yl)-quinoxaline (40 mg, 0.14 mmol). Evaporation of solvent from the organic extract yielded 30 mg of a crude product, which was further purified by preparative thin layer chromatography to give 10 mg (27%) of the title compound. MS (electrospray): mass calculated for C₁₃H₁₅FN₄O, 262.3; m/z found, 263.3 [M+H]⁺. ¹H NMR (400 MHz, DMSO-d₆): 10.58 (brs, 1H), 7.21-7.18 (dd, J=9.6, 2.8 Hz, 1H), 7.04-7.00 (dd, J=9.1, 5.8 Hz, 1H), 6.96-6.91 (m, 1H), 4.13-4.10 (m, 4H), 2.59-2.56 (m, 4H), 2.36 (s, 3H).

### Example 22

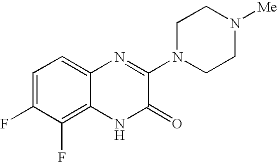 7,8-Difluoro-3-(4-methyl-piperazin-1-yl)-1H-quinoxalin-2-one.

A. 7,8-Difluoro-3-methoxy-1H-quinolin-2-one. The reaction was carried out as described in General Procedure 1 using 3,4-trifluoromethyl-1,2-phenylenediamine (680 mg, 4.68 mmol). After cooling to room temperature, the solution was concentrated in vacuo and used without further purification. MS (electrospray): mass calculated for C₁₀H₇F₂N₂O, 211.0; m/z found, 212.3 [M+H]⁺. ¹H NMR (400 MHz, CDCl₃): 12.72 (brs, 1H), 7.34-7.28 (m, 1H), 7.25-7.21 (m, 1H), 4.00 (s, 3H).

B. 7,8-Difluoro-3-(4-methyl-piperazin-1-yl)-1H-quinoxalin-2-one. The reaction was carried out as described in General Procedure 2 with 7,8-difluoro-3-methoxy-1H-quinolin-2-one (100 mg, 0.47 mmol). Purification by silica gel chromatography (0-5% MeOH/DCM) afforded 40 mg (31%) of the title compound. MS (electrospray): mass calculated for C₁₃H₁₄F₂N₄O, 280.1; m/z found, 281.3 [M+H]⁺. ¹H NMR (400 MHz, CDCl₃): 9.45 (br s, 2H), 7.23-7.19 (m, 1H), 7.02-6.98 (m, 1H), 4.04-4.02 (m, 4H), 2.58-2.55 (m, 4H), 2.35 (s, 3H).

### Example 23

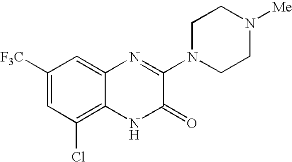 8-Chloro-3-(4-methyl-piperazin-1-yl)-6-trifluoromethyl-1H-quinoxalin-2-one.

A. 8-Chloro-3-methoxy-6-trifluoromethyl-1H-quinoxalin-2-one. The reaction was carried out as described in General Procedure 1 using 3-chloro-5-trifluoromethyl-1,2-phenylenediamine (2.0 mg, 9.50 mmol). After the reaction mixture cooled to room temperature, 1.5 g of precipitate was collected by filtration and used without further purification. MS (electrospray): mass calculated for C₁₀H₆ClF₃N₂O₂, 278.0; m/z found, 279.0 [M+H]⁺. ¹H NMR (400 MHz, CD₃OD): 7.81 (s, 1H), 7.73 (s, 1H), 4.11 (s, 3H).

B. 8-Chloro-3-(4-methyl-piperazin-1-yl)-6-trifluoromethyl-1H-quinoxalin-2-one. The reaction was carried out as described in General Procedure 2 with 8-chloro-3-methoxy-6-trifluoromethyl-1H-quinoxalin-2-one (165 mg, 0.59 mmol). Purification by silica gel chromatography (0-5% MeOH/DCM) afforded 150 mg (73%) of the title compound. MS (electrospray): mass calculated for C₁₄H₁₄ClF₃N₄O, 346.1; m/z found, 347.2 [M+H]⁺. ¹H NMR (400 MHz, CDCl₃): 8.93 (br s, 1H), 7.65 (s, 1H), 7.44 (s, 1H), 4.15-4.12 (m, 4H), 2.56-2.53 (m, 4H), 2.30 (s, 3H).

### Example 24

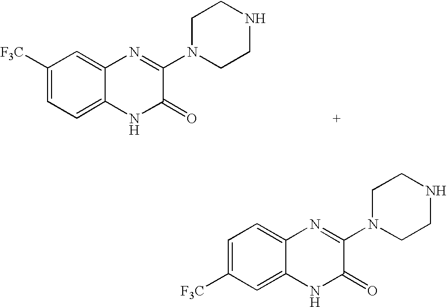 3-piperazin-1-yl-6-trifluoromethyl-1H-quinoxalin-2-one; and 3-piperazin-1-yl-7-trifluoromethyl-1H-quinoxalin-2-one.

The reaction was carried out as described in General Procedure 3 with a 1:1 mixture of 3-methoxy-6-trifluoromethyl-1H-quinoxalin-2-one and 3-methoxy-7-trifluoromethyl-1H-quinoxalin-2-one (100 mg, 0.41 mmol total) and piperazine (176 mg, 2.05 mmol). Purification by reversed-phase chromatography (Cl₈; 10-90% MeOH/MeCN, 1% TFA) gave the product as the TFA salt (10 mg, 8%) of a 1:1 mixture of the title regioisomers. MS (electrospray): mass calculated for C₁₃H₁₃F₃N₄O, 298.2; m/z found, 299.1 [M+H]⁺. ¹H NMR (400 MHz, acetone-d₆): 7.57-7.55 (m, 1H), 7.46-7.43 (m, 1H), 7.39-7.31 (m, 4H), 4.30-4.26 (m, 4H), 3:41-3.38 (m, 4H).

The following mixtures of regioisomers in Examples 25-28 were prepared from the appropriate starting materials by following General Procedures 1 and 2. The mixtures were purified by reversed-phase chromatography (C₁₈; 10-90% MeOH/MeCN, 1% TFA), which gave the products as the TFA salts. All NMR spectra were consistent with 1:1 mixtures of regioisomers, as in Example 11. Individual regioisomers can be prepared from the appropriate quinoxaline-diones by following General Procedures 4-6.

### Example 25

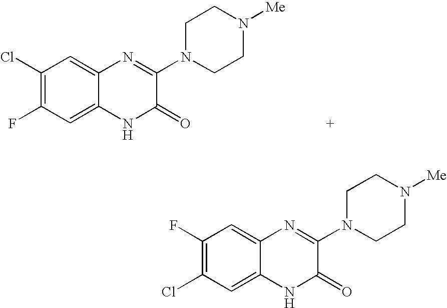 6-Chloro-7-fluoro-3-(4-methyl-piperazin-1-yl)-1H-quinoxalin-2-one; and 7-Chloro-6-fluoro-3-(4-methyl-piperazin-1-yl)-1H-quinoxalin-2-one. MS (electrospray): mass calculated for C₁₃H₁₄ClFN₄O, 296.1; m/z found, 297.2 [M+H]⁺ (single peak observed).

### Example 26

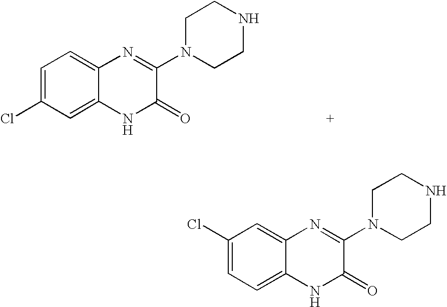 7-Chloro-3-piperazin-1-yl-1H-quinoxalin-2-one; and 6-Chloro-3-piperazin-1-yl-1H-quinoxalin-2-one.

MS (electrospray): mass calculated for C₁₂H₁₃ClN₄O, 264.0; m/z found, 265.0 [M+H]⁺ (single peak observed).

### Example 27

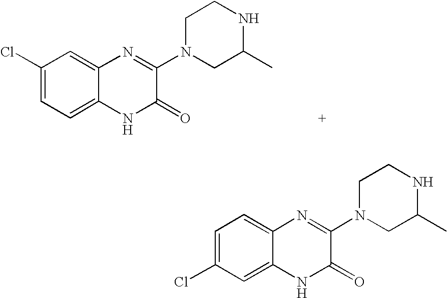 6-Chloro-3-(3-methyl-piperazin-1-yl)-1H-quinoxalin-2-one; and 7-Chloro-3-(3-methyl-piperazin-1-yl)-1H-quinoxalin-2-one.

MS (electrospray): mass calculated for C₁₃H₁₅ClN₄O, 278.1; m/z found, 279.1 [M+H]⁺ (single peak observed).

### Example 28

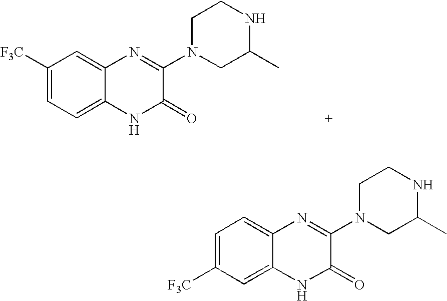

3-(3-Methyl-piperazin-1-yl)-6-trifluoromethyl-1H-quinoxalin-2-one; and 3-(3-Methyl-piperazin-1-yl)-7-trifluoromethyl-1H-qui noxalin-2-one.

MS (electrospray): mass calculated for C₁₄H₁₅F₃N₄O, 312.1; m/z found, 313.2 [M+H]⁺ (single peak observed).

### Biological Examples

Binding Assay on Recombinant Human Histamine H₄ Receptor

SK-N-MC cells or COS7 cells were transiently transfected with pH4R and grown in 150 cm² tissue culture dishes. Cells were washed with saline solution, scraped with a cell scraper and collected by centrifugation (1000 rpm, 5 min). Cell membranes were prepared by homogenization of the cell pellet in 20 mM Tris-HCl with a polytron tissue homogenizer for 10 s at high speed. Homogenate was centrifuged at 1000 rpm for 5 min at 4° C. The supernatant was then collected and centrifuged at 20,000× g for 25 min at 4° C. The final pellet was resuspended in 50 mM Tris-HCl. Cell membranes were incubated with ³H-histamine (5-70 nM) in the presence or absence of excess histamine (10000 nM). Incubation occurred at room temperature for 45 min. Membranes were harvested by rapid filtration over Whatman GF/C filters and washed 4 times with ice-cold 50 mM Tris HCl. Filters were then dried, mixed with scintillant and counted for radioactivity. SK-N-MC or COS7 cells expressing human histamine H₄ receptor were used to measure the affinity of binding of other compounds and their ability to displace ³H-ligand binding by incubating the above-described reaction in the presence of various concentrations of inhibitor or compound to be tested. For competition binding studies using ³H-histamine, Kᵢ values were calculated, based on an experimentally determined K_{D} value of 5 nM and a ligand concentration of 5 nM, according to Y.-C. Cheng and W. H. Prusoff (Biochem. Pharmacol. 1973, 22(23):3099-3108): Kᵢ=(IC₅₀)/(1+([L]/(K_{D})). Data are shown in Table 1.

Binding Assay Results

TABLE 1

| EX | Kᵢ (nM) |
|---|---|
| 1 | 6 |
| 2 | 16 |
| 3 | 640 |
| 4 | 430 |
| 5 | 40 |
| 6 | 681 |
| 7 | 852 |
| 8 | 21 |
| 9 | 84 |
| 10 | 31 |
| 11 | 11 |
| 12 | 24 |
| 13 | 19 |
| 14 | 32 |
| 15 | 56 |
| 16 | 1610 |
| 17 | 74 |
| 18 | 112 |
| 19 | 93 |
| 20 | 83 |
| 21 | 39 |
| 22 | 30 |
| 23 | 166 |
| 24 | 467 |
| 25 | 12 |
| 26 | 152 |
| 27 | 467 |
| 28 | 743 |

Mast Cell Chemotaxis Assay

Mast cell accumulation in mucosal epithelia is a well-known characteristic of allergic rhinitis and asthma. In addition, it is known that mast cell numbers increase in a number of inflammatory conditions. Some of this is due to chemotaxis of mast cells to the sites of inflammation. This chemotaxis to specific agents can be mimicked in vitro. Transwells (Costar, Cambridge, Mass.) of a pore size 8 μm were coated with 100 μL of 100 ng/mL human fibronectin (Sigma) for 2 h at room temperature. After removal of the fibronectin, 600 μL of RPMI with 5% BSA, in the presence of 10 μM histamine, was added to the bottom chamber. To test the various histamine receptor (HR) antagonists, 10 μM and/or 1 μM solutions of the test compounds were added to the top and bottom chambers. Mast cells (2×10⁵/well) were added to the top chamber. The plates were incubated for 3 h at 37° C. Transwells were removed and the cells in the bottom chamber were counted for sixty seconds using a flow cytometer.

| 10 μM | HR Antagonist (μM): | | Binding |
|---|---|---|---|
| Histamine | 10 | 1 | Assay |
| EX | % Inh | % Inh | Kᵢ (nM) |
| 1 | | 76 | 6 |
| 2 | | 50 | 16 |
| 12 | | 99 | 24 |
| 14 | 80 | 71 | 32 |
| 25 | 79 | 81 | 12 |

Cell-type Distribution of H₄ Expression

RNA was prepared from the different cells using an RNeasy kit (Qiagen, Valencia, Calif.) according to the manufacturer's instructions. Total RNA was extracted from purified human cells using the RNeasy kit (Qiagen, Valencia, Calif.) and reverse transcribed to cDNA using the RT reaction kit (Invitrogen) according to the manufacturer's instructions. H₄ receptor RNA was detected by RT-PCR using human H₄ receptor-specific primers 5′-ATGCCAGATACTAATAGCACA and 5′-CAGTCGGTCAGTATCTTCT. The amplified PCR band for H₄ receptor is 1170 bp.

Results

The RT-PCR results indicate that the H₄ receptor is expressed on mast cells, dendritic cells, basophils, and eosinophils. These positive results are consistent with the published literature (e.g. Oda et al., Nguyen et al., and Morse et al. in the Background section). Accumulation of mast cells and eosinophils in affected tissues is one of the principal characteristics of allergic rhinitis and asthma. Since H₄ receptor expression is found in these cell types; H₄ receptor signalling is likely to mediate the infiltration of mast cells and eosinophils in response to histamine. The following table reports the Cell-type Distribution of H₄ Expression by RT-PCR.

| Species | Cell Type | H₄ |
|---|---|---|
| Human | Eosinophils | + |
| | Immature Dendritic Cells | + |
| | Mature Dendritic Cells | + |
| | Mast Cells | + |
| | Basophils | + |
| | CD14⁺ Monocytes | − |
| | CD4⁺ T Cells | + |
| | CD8⁺ T Cells | − |
| | B Cells | − |
| | Neutrophils | − |
| Mouse/(Rat) | Eosinophils | + |
| | Peritoneal Mast Cells (Rat) | + |
| | Bone Marrow-Derived | + |
| | Mast Cells | |
| | Immature Dendritic Cells | + |
| | Mature Dendritic Cells | + |
| | Bone Marrow-Derived | − |
| | Macrophages | |
| | Peritoneal Macrophages | − |
| | CD4⁺ T Cells | − |
| | CD8⁺ T Cells | − |
| | B Cells | − |

The Inhibition of Eosinophil Shape Change by Histamine H₄ Receptor Antagonists

Eosinophil accumulation in sites of allergic reaction is a well-known characteristic of allergic rhinitis and asthma. This example demonstrates that histamine H₄ receptor antagonists can block the shape change response in human eosinophils in response to histamine. Shape change is a cellular characteristic that precedes eosinophil chemotaxis.

Methods

Human granulocytes were isolated from human blood by a Ficoll gradient. The red blood cells were lysed with 5-10× Qiagen lysis buffer at room temperature for 5-7 min. Granulocytes were harvested and washed once with FACS buffer. The cells were resuspended at a density of 2×10⁶ cells/mL in reaction buffer. To test inhibition by specific histamine receptor antagonists, 90 μL of the cell suspension (˜2×10⁵ cells) was incubated with 10 μM of one of the various test compound solutions. After 30 min, 11 μL of one of the various concentrations of histamine was added. Ten minutes later the cells were transferred to ice and fixed with 250 μL of ice-cold fixative buffer (2% formaldehyde) for 1 min. The shape change was quantitated using a gated autofluoescence forward scatter assay (GAFS) (S. A. Bryan et al., Am. J. Respir. Crit. Care Med. 2002, 165(12):1602-1609).

Results—Histamine Mediates Eosinophil Shape Change Through H₄ Receptor

The change in shape of eosinophils is due to cytoskeletal changes that preceed chemotaxis and thus is a measure of chemotaxis. The data in the following table show that histamine induces a dose-dependent shape change in eosinophils. Histamine receptor (HR) antagonists were used to sort out which histamine receptor is responsible for the shape change. Antagonists specific for the histamine H₁ receptor (diphenhydramine) or the H₂ receptor (ranatidine) did not alter the histamine-induced shape change. However, a dual H₃/H₄ antagonist (thioperamide) and a specific histamine H₄ receptor antagonist ((5-chloro-1H-indol-2-yl)-(4-methyl-piperazin-1-yl)-methanone, Kᵢ=5 nM) inhibited histamine-induced eosinophil shape change with an IC₅₀ of 1.5 and 0.27 μM, respectively.

| | Fold Change | | | | |
|---|---|---|---|---|---|
| Histamine | 10 | 1 | 0.1 | 0.01 | 0 |
| (μM): | | | | | |
| No HR | 1.34 | 1.31 | 1.21 | 1.01 | 1.00 |
| Antagonist | | | | | |
| 10 μM H₄ | 1.09 | 1.05 | 1.05 | 1.01 | 1.00 |
| Antagonist | | | | | |
| 10 μM | 1.08 | 1.05 | 1.01 | 1.04 | 1.00 |
| Thiop | | | | | |
| 10 μM | 1.63 | 1.50 | 1.18 | 1.03 | 1.00 |
| Diphen | | | | | |
| 10 μM | 1.64 | 1.49 | 1.21 | 1.04 | 1.00 |
| Ranat | | | | | |

The Inhibition of Eosinophil Chemotaxis by Histamine H₄ Receptor Antagonists

Eosinophil accumulation in sites of allergic reaction is a well-known characteristic of allergic rhinitis and asthma. Eosinophils are purified from human blood with standard methods. Chemotaxis assays are carried out using transwells (Costar, Cambridge, Mass.) of a pore size 5 μm coated with 100 μL of 100 ng/mL human fibronectin (Sigma) for 2 h at room temperature. After removal of the fibronectin, 600 μL of RPMI with 5% BSA in the presence of histamine (ranging from 1.25-20 μM) is added to the bottom chamber. To test the various histamine receptor antagonists 10 μM of the test compounds can be added to the top and bottom chambers. Eosinophils will be added to the top chamber whereas histamine or chemotactic factors will be placed in the lower chamber. The plates are incubated for 3 h at 37° C. Transwells are removed and the number of cells in the bottom chamber can be counted for 60 s using a flow cytometer, or can be quantitated by using Giemsa staining.

The Inhibition of Zymosan-Induced Peritonitis in Mice by Histamine H₄ Receptor Antagonists

It has been demonstrated that histamine H₄ receptor antagonists can block the peritonitis induced by zymosan, which is the insoluble polysaccharide component on the cell wall of Saccharomyces cerevisiae. This is commonly used to induce peritonitis in mice and appears to act in a mast cell-dependent manner. Compounds of the present invention can be tested in such a model to demonstrate their use as anti-inflammatory agents. At time 0 mice are given compound or PBS, either s.c. or p.o. Fifteen minutes later each mouse receives 1 mg zymosan A (Sigma) i.p. The mice are sacrificed 4 h later, and the peritoneal cavities are washed with 3 mL of PBS containing 3 mM EDTA. The number of migrated leukocytes is determined by taking an aliquot (100 μL) of the lavage fluid and diluting 1:10 in Turk's solution (0.01% crystal violet in 3% acetic acid). The samples are then vortexed, and 10 μL of the stained cell solution is placed in a Neubauer haemocytometer. Differential cell counts are performed using a light microscope (Olympus B061). In view of their chromatic characteristics and their nucleus and cytoplasm appearance, polymorphonuclear leukocytes (PMN; >95% neutrophils) can be easily identified. Treatment with zymosan increases the number of neutrophils, which is representative of an inflammatory response. Treatment with H₄ receptor antagonist blocks this incease.

Inhibition of Mast Cell Chemotaxis by H₄ Receptor Antagonist in an Animal Model of Asthma and Allergic Rhinitis

An animal model is used to test the observation that mast cells accumulate in response to allergic inflammation and that this can be blocked by H₄ receptor antagonists. Compounds of the present invention can be tested in this model to demonstrate their use as treatments for allergic rhinitis or asthma. Mice are be sensitized by intraperitoneal injection of ovalbumin/Alum (10 μg in 0.2 ml Al(OH)₃; 2%) on Day 0 and Day 14. On Day 21 through 23 mice are challenged by PBS or ovalbumin, and sacrificed 24 h after the last challenge on Day 24. A section of the trachea is removed and fixed in formalin. Paraffin embedding and longitudinal sectioning of tracheas are performed followed by staining of mast cells with toluidine blue. Alternatively, trachea are frozen in OCT for frozen sectioning, and mast cells are identified by IgE staining. Mast cells are quantified as sub-mucosal or sub-epithelial depending on their location within each tracheal section. Exposure to allergen should increase the number of sub-epithelial mast cells, and this effect is blocked by H₄ receptor antagonists.

The features and advantages of the invention are apparent to one of ordinary skill in the art. Based on this disclosure, including the summary, detailed description, background, examples, and claims, one of ordinary skill in the art will be able to make modifications and adaptations to various conditions and usages. Publications described herein are incorporated by reference in their entirety. These other embodiments are also within the scope of the invention.

1. A pharmaceutical composition for treating or preventing an H₄ receptor-mediated condition in a subject, comprising a therapeutically effective amount, said therapeutically effective amount being effective for treating or preventing an H₄ receptor-mediated condition, of at least one of an H₄ receptor modulator of formula (I): 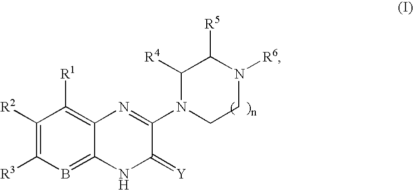
wherein
B is, independently from other member and substituent assignments, N or CR⁷;
Y is, independently from other member and substituent assignments, O, S or NH;
n is, independently from member and substituent assignments, 1 or 2;
each of the substituents R¹⁻³ and R⁷ is, independently from other member and substituent assignments, H, F, Cl, Br, I, C₁₋₄ alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of R^{a}, and R^{b} is, independently from other substituent assignments, selected from H, C₁₋₄alkyl, phenyl, benzyl or phenethyl, and wherein any phenyl, alkyl, and cycloalkyl moiety in any of said R¹⁻³, R⁷, R^{a}, and Rb, is optionally, and independently from other substituent assignments, substituted with 1 to 3 substituents selected from C₁₋₃alkyl, halo, hydroxy, amino, and C₁₋₃alkoxy;
each of R and R⁵ is, independently from other member and substituent assignments, H or C₁₋₆alkyl;
R⁶ is, independently from other member and substituent assignments, H, C₁₋₆ alkyl, C₃₋₅alkenyl with no sp²-carbon member attached directly to the R⁶-attached nitrogen member, C₃₋₅alkynyl with no sp-carbon member attached directly to the R⁶-attached nitrogen member, CH₂CH₂OH, or —Cl₄alkyl-O—C₁₋₄alkyl;
alternatively, R⁶ may be taken together with R⁵, the R⁵-attached carbon member, and the R⁶-attached nitrogen member to form a 5-, 6- or 7-membered heterocyclic ring HetCyc1, wherein said ring HetCyc1 has 0 or 1 additional heteroatoms selected from O, S, >NH or >NC₁₋₆alkyl, and wherein said ring HetCycl is substituted with 0, 1, 2 or 3 substituents each selected independently from other substituent assignments from C₁₋₃alkyl, halo, hydroxy, amino, and C₁₋₃alkoxy;
an enantiomer, diastereomer, racemate thereof, and pharmaceutically acceptable salt and ester thereof.

2. A composition as in claim 1, wherein B is CR⁷.

3. A composition as in claim 1, wherein Y is O.

4. A composition as in claim 1, wherein n is 1.

5. A composition as in claim 1, wherein each of R¹⁻³ and R⁷ is, independently from other member and substituent assignments, selected from the group consisting of H, —F, —Cl, —Br, —I, —CH₃, —CH₂CH₃, —OCH₃, —OCH₂CH₃, —OCH(CH₃)₂, cyclopropyl, cyclobutyl, cyclopentyl, cyclohexyl, -Ocyclopentyl, -Ocyclohexyl, —CF₃, —OCF₃, —SCF₃, —OH, —NO₂, —NH₂, —NHCH₃, —N(CH₃)₂, —N(CH₂CH₃)₂, —CN and phenyl.

6. A composition as in claim 1, wherein R¹⁻³ and R⁷ are independently selected from the group consisting of hydrogen, methyl, trifluoromethyl, methoxy, trifluoromethoxy, nitro, chloro, and fluoro.

7. A composition as in claim 1, wherein one or two of R¹⁻³ and R⁷ are not hydrogen.

8. A composition as in claim 1, wherein R⁴ and R⁵ are, independently, selected from the group consisting of
a) H, and
b) —CH₃, —CH₂CH₃, —CH₂CH₂CH₃, —CH(CH₃)₂, n-butyl, i-butyl, t-butyl.

9. A composition as in claim 1, wherein R⁴ and R⁵ are independently H or —CH₃.

10. A composition as in claim 1, wherein R⁶ is selected from the group consisting of
a) H,
b) CH₂CH₂OH, and
c) —CH₃, —CH₂CH₃, —CH₂CH₂CH₃, —CH(CH₃)₂, n-butyl, i-butyl, t-butyl, —CH₂CH₂OCH₃, —CH₂CH₂OCH₂CH₃, —CH₂CH₂OCH₂CH₂CH₃, —CH₂CH₂OCH(CH₃)₂, —CH₂CH₂O-n-butyl, —CH₂CH₂O-1-butyl, and —CH₂CH₂O-t-butyl.

11. A composition as in claim 1, wherein R⁵ is selected from the group consisting of H, —CH₃, and —CH₂CH₃.

12. A composition as in claim 1, wherein R⁶ taken together with an adjacent R⁵ as well as their carbon and nitrogen of attachment is selected from the group consisting of pyrrolidin-1,2-yl, pyrazolidin-1,5-yl, piperidin-1,2-yl, piperazin-1,2-yl, morpholin-4,5-yl and thiomorpholin-4,5-yl.

13. A composition as in claim 1, wherein R⁶ taken together with an adjacent R⁵ as well as their carbon and nitrogen of attachment is pyrrolidin-1,2-yl or piperidin-1,2-yl.

14. A pharmaceutical composition for inhibiting leukocyte recruitment in a subject, comprising a therapeutically effective amount, said therapeutically effective amount being effective for inhibiting leukocyte recruitment in a subject, of at least one of a leukocyte recruitment inhibitor of formula (I): 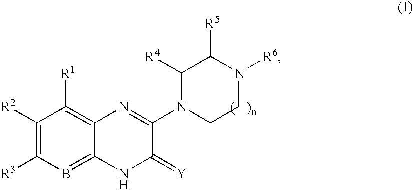
wherein
B is, independently from other member and substituent assignments, N or CR⁷;
Y is, independently from other member and substituent assignments, O, S or NH;
n is, independently from member and substituent assignments, 1 or 2;
each of the substituents R¹⁻³ and R⁷ is, independently from other member and substituent assignments, H, F, Cl, Br, I, C₁₋₄ alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of R^{a}, and R^{b} is, independently from other substituent assignments, selected from H, C₁₋₄alkyl, phenyl, benzyl or phenethyl, and wherein any phenyl, alkyl, and cycloalkyl moiety in any of said R¹⁻³, R⁷, R^{a}, and R^{b}, is optionally, and independently from other substituent assignments, substituted with 1 to 3 substituents selected from C₁₋₃alkyl, halo, hydroxy, amino, and C₁₋₃alkoxy;
each of R⁴ and R⁵ is, independently from other member and substituent assignments, H or C₁₋₆alkyl;
R⁶ is, independently from other member and substituent assignments, H, C-6 alkyl, C₃₋₅alkenyl with no sp²-carbon member attached directly to the R⁶-attached nitrogen member, C₃₋₅alkynyl with no sp-carbon member attached directly to the R 6-attached nitrogen member, CH₂CH₂OH, or —Cl₄alkyl-O—C₁₋₄alkyl;
alternatively, R⁵ may be taken together with R⁵, the R⁵-attached carbon member, and the R⁶-attached nitrogen member to form a 5-, 6- or 7-membered heterocyclic ring HetCyc1, wherein said ring HetCycl has 0 or 1 additional heteroatoms selected from O, S, >NH or >NC₁₋₆alkyl, and wherein said ring HetCycl is substituted with 0, 1, 2 or 3 substituents each selected independently from other substituent assignments from C₁₋₃alkyl, halo, hydroxy, amino, and C₁₋₃alkoxy;
an enantiomer, diastereomer, racemate thereof, and pharmaceutically acceptable salt and ester thereof.

15. A composition as in claim 14, wherein B is CR⁷.

16. A composition as in claim 14, wherein Y is O.

17. A composition as in claim 14, wherein n is 1.

18. A composition as in claim 14, wherein each of R¹⁻³ and R⁷ is, independently from other member and substituent assignments, selected from the group consisting of H, —F, —Cl, —Br, —I, —CH₃, —CH₂CH₃, —OCH₃, —OCH₂CH₃, —OCH(CH₃)₂, cyclopropyl, cyclobutyl, cyclopentyl, cyclohexyl, -Ocyclopentyl, -Ocyclohexyl, —CF₃, —OCF₃, —SCF₃, —OH, —NO₂, —NH₂, —NHCH₃, —N(CH₃)₂, —N(CH₂CH₃)₂, —CN and phenyl.

19. A composition as in claim 14, wherein R¹⁻³ and R⁷ are independently selected from the group consisting of hydrogen, methyl, trifluoromethyl, methoxy, trifluoromethoxy, nitro, chloro, and fluoro.

20. A composition as in claim 14, wherein one or two of R¹⁻³ and R⁷ are not hydrogen.

21. A composition as in claim 14, wherein R⁴ and R⁵ are, independently, selected from the group consisting of
a) H, and
b) —CH₃, —CH₂CH₃, —CH₂CH₂CH₃, —CH(CH₃)₂, n-butyl, i-butyl, t-butyl.

22. A composition as in claim 14, wherein R⁴ and R⁵ are independently H or —CH₃.

23. A composition as in claim 14, wherein R⁶ is selected from the group consisting of
a) H,
b) CH₂CH₂OH, and
c) —CH₃, —CH₂CH₃, —CH₂CH₂CH₃, —CH(CH₃)₂, n-butyl, i-butyl, t-butyl, —CH₂CH₂OCH₃, —CH₂CH₂OCH₂CH₃, —CH₂CH₂OCH₂CH₂CH₃, —CH₂CH₂OCH(CH₃)₂, —CH₂CH₂O-n-butyl, —CH₂CH₂O-1-butyl, and —CH₂CH₂O-t-butyl.

24. A composition as in claim 14, wherein R⁶ is selected from the group consisting of H, —CH₃, and —CH₂CH₃.

25. A composition as in claim 14, wherein R⁶ taken together with an adjacent R⁵ as well as their carbon and nitrogen of attachment is selected from the 15 group consisting of pyrrolidin-1,2-yl, pyrazolidin-1,5-yl, piperidin-1,2-yl, piperazin-1,2-yl, morpholin-4,5-yl and thiomorpholin-4,5-yl.

26. A composition as in claim 14, wherein R⁶ taken together with an adjacent R⁵ as well as their carbon and nitrogen of attachment is pyrrolidin-1,2-yl or piperidin-1,2-yl.

27. An anti-inflammatory composition, comprising a therapeutically effective amount, said therapeutically effective amount being effective for treating or preventing inflammation, of at least one of anti-inflammatory compound of formula (I): 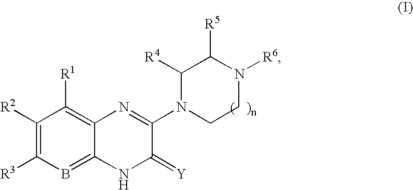
wherein
B is, independently from other member and substituent assignments, N or CR⁷;
Y is, independently from other member and substituent assignments, O, S or NH;
n is, independently from member and substituent assignments, 1 or 2;
each of the substituents R¹⁻³ and R⁷ is, independently from other member and substituent assignments, H, F, Cl, Br, I, C₁₋₄ alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of R^{a}, and R^{b} is, independently from other substituent assignments, selected from H, C₁₋₄alkyl, phenyl, benzyl or phenethyl, and wherein any phenyl, alkyl, and cycloalkyl moiety in any of said R¹⁻³, R⁷, R^{a}, and R^{b}, is optionally, and independently from other substituent assignments, substituted with 1 to 3 substituents selected from C₁₋₃alkyl, halo, hydroxy, amino, and C₁₋₃alkoxy;
each of R⁴ and R⁵ is, independently from other member and substituent assignments, H or C₁₋₆alkyl;
R⁶ is, independently from other member and substituent assignments, H, C₁₋₆ alkyl, C₃₋₅alkenyl with no sp²-carbon member attached directly to the R⁶-attached nitrogen member, C₃₋₅alkynyl with no sp-carbon member attached directly to the R⁵-attached nitrogen member, CH₂CH₂OH, or —C₁₋₄alkyl-O—C₁₋₄alkyl;
alternatively, R may be taken together with R⁵, the R⁵-attached carbon member, and the R⁶-attached nitrogen member to form a 5-, 6- or 7-membered heterocyclic ring HetCyc1, wherein said ring HetCyc1 has 0 or 1 additional heteroatoms selected from O, S, >NH or >NC₁₋₆alkyl, and wherein said ring HetCycl is substituted with 0, 1, 2 or 3 substituents each selected independently from other substituent assignments from C₁₋₃alkyl, halo, hydroxy, amino, and C₁₋₃alkoxy;
an enantiomer, diastereomer, racemate thereof, and pharmaceutically acceptable salt and ester thereof.

28. A composition as in claim 27, wherein B is CR⁷.

29. A composition as in claim 27, wherein Y is O.

30. A composition as in claim 27, wherein n is 1.

31. A composition as in claim 27, wherein each of R¹⁻³ and R⁷ is, independently from other member and substituent assignments, selected from the group consisting of H, —F, —Cl, —Br, —I, —CH₃, —CH₂CH₃, —OCH₃, —OCH₂CH₃, —OCH(CH₃)₂, cyclopropyl, cyclobutyl, cyclopentyl, cyclohexyl, -Ocyclopentyl, —Ocyclohexyl, —CF₃, —OCF₃, —SCF₃, —OH, —NO₂, —NH₂, —NHCH₃, —N(CH₃)₂, —N(CH₂CH₃)₂, —CN and phenyl.

32. A composition as in claim 27, wherein R¹⁻³ and R⁷ are independently selected from the group consisting of hydrogen, methyl, trifluoromethyl, methoxy, trifluoromethoxy, nitro, chloro, and fluoro.

33. A composition as in claim 27, wherein one or two of R¹⁻³ and R⁷ are not hydrogen.

34. A composition as in claim 27, wherein R⁴ and R⁵ are, independently, selected from the group consisting of
a) H, and
b) —CH₃, —CH₂CH₃, —CH₂CH₂CH₃, —CH(CH₃)₂, n-butyl, i-butyl, t-butyl.

35. A composition as in claim 27, wherein R⁴ and R⁵ are independently H or —CH₃.

36. A composition as in claim 27, wherein R⁶ is selected from the group consisting of
a) H,
b) CH₂CH₂OH, and
c) —CH₃, —CH₂CH₃, —CH₂CH₂CH₃, —CH(CH₃)₂, n-butyl, i-butyl, t-butyl, —CH₂CH₂OCH₃, —CH₂CH₂OCH₂CH₃, —CH₂CH₂OCH₂CH₂CH₃, —CH₂CH₂OCH(CH₃)₂, —CH₂CH₂O-n-butyl, —CH₂CH₂O-1-butyl, and —CH₂CH₂O-t-butyl.

37. A composition as in claim 27, wherein R⁶ is selected from the group consisting of H, —CH₃, and —CH₂CH₃.

38. A composition as in claim 27, wherein R⁶ taken together with an adjacent R⁵ as well as their carbon and nitrogen of attachment is selected from the group consisting of pyrrolidin-1,2-yl, pyrazolidin-1,5-yl, piperidin-1,2-yl, piperazin-1,2-yl, morpholin-4,5-yl and thiomorpholin-4,5-yl.

39. A composition as in claim 27, wherein R⁶ taken together with an adjacent R⁵ as well as their carbon and nitrogen of attachment is pyrrolidin-1,2-yl or piperidin-1,2-yl.

40. A method for treating or preventing inflammation in a subject, comprising administering to the subject in connection with an inflammatory response a pharmaceutical composition that comprises at least one of a therapeutically effective amount of an anti-inflammatory compound of formula (I): 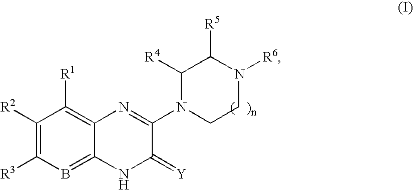
wherein
B is, independently from other member and substituent assignments, N or CR⁷;
Y is, independently from other member and substituent assignments, O, S or NH;
n is, independently from member and substituent assignments, 1 or 2;
each of the substituents R¹⁻³ and R⁷ is, independently from other member and substituent assignments, H, F, Cl, Br, I, C₁₋₄ alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of R^{a}, and R^{b} is, independently from other substituent assignments, selected from H, C₁₋₄alkyl, phenyl, benzyl or phenethyl, and wherein any phenyl, alkyl, and cycloalkyl moiety in any of said R¹⁻³, R⁷, R^{a}, and R^{b}, is optionally, and independently from other substituent assignments, substituted with 1 to 3 substituents selected from C₁₋₃alkyl, halo, hydroxy, amino, and C₁₋₃alkoxy;
each of R⁴ and R⁵ is, independently from other member and substituent assignments, H or C₁₋₆alkyl;
R⁶ is, independently from other member and substituent assignments, H, C₁₋₆ alkyl, C₃₋₅alkenyl with no sp²-carbon member attached directly to the R⁶-attached nitrogen member, C₃₋₅alkynyl with no sp-carbon member attached directly to the R⁶-attached nitrogen member, CH₂CH₂OH, or —C₁₋₄alkyl-O—C₁₋₄alkyl;
alternatively, R⁵ may be taken together with R⁵, the R⁵-attached carbon member, and the R⁶-attached nitrogen member to form a 5-, 6- or 7-membered heterocyclic ring HetCyc1, wherein said ring HetCyc1 has 0 or 1 additional heteroatoms selected from O, S, >NH or >NC₁₋₆alkyl, and wherein said ring HetCyc1 is substituted with 0, 1, 2 or 3 substituents each selected independently from other substituent assignments from C₁₋₃alkyl, halo, hydroxy, amino, and C₁₋₃alkoxy;
an enantiomer, diastereomer, racemate thereof, and pharmaceutically acceptable salt and ester thereof.

41. A method as in claim 40, wherein B is CR⁷.

42. A method as in claim 40, wherein Y is O.

43. A method as in claim 40, wherein n is 1.

44. A method as in claim 40, wherein each of R¹⁻³ and R⁷ is, independently from other member and substituent assignments, selected from the group consisting of H, —F, —Cl, —Br, —I, —CH₃, —CH₂CH₃, —OCH₃, —OCH₂CH₃, —OCH(CH₃)₂, cyclopropyl, cyclobutyl, cyclopentyl, cyclohexyl, —Ocyclopentyl, —Ocyclohexyl, —CF₃, —OCF₃, —SCF₃, —OH, —NO₂, —NH₂, —NHCH₃, —N(CH₃)₂, —N(CH₂CH₃)₂, —CN and phenyl.

45. A method as in claim 40, wherein R¹⁻³ and R⁷ are independently selected from the group consisting of hydrogen, methyl, trifluoromethyl, methoxy, trifluoromethoxy, nitro, chloro, and fluoro.

46. A method as in claim 40, wherein one or two of R¹⁻³ and R⁷ are not hydrogen.

47. A method as in claim 40, wherein R⁴ and R⁵ are, independently, selected from the group consisting of
a) H, and
b) —CH₃, —CH₂CH₃, —CH₂CH₂CH₃, —CH(CH₃)₂, n-butyl, i-butyl, t-butyl.

48. A method as in claim 40, wherein R⁴ and R⁵ are independently H or —CH₃.

49. A method as in claim 40, wherein R⁵ is selected from the group consisting of
a) H,
b) CH₂CH₂OH, and
c) —CH₃, —CH₂CH₃, —CH₂CH₂CH₃, —CH(CH₃)₂, n-butyl, i-butyl, t-butyl, —CH₂CH₂OCH₃, —CH₂CH₂OCH₂CH₃, —CH₂CH₂OCH₂CH₂CH₃, —CH₂CH₂OCH(CH₃)₂, —CH₂CH₂O-n-butyl, —CH₂CH₂O-1-butyl, and —CH₂CH₂O-t-butyl.

50. A method as in claim 40, wherein R⁶ is selected from the group consisting of H, —CH₃, and —CH₂CH₃.

51. A method as in claim 40, wherein R⁶ taken together with an adjacent R⁵ as well as their carbon and nitrogen of attachment is selected from the group consisting of pyrrolidin-1,2-yl, pyrazolidin-1,5-yl, piperidin-1,2-yl, piperazin-1,2-yl, morpholin-4,5-yl and thiomorpholin-4,5-yl.

52. A method as in claim 40, wherein R⁶ taken together with an adjacent R⁵ as well as their carbon and nitrogen of attachment is pyrrolidin-1,2-yl or piperidin-1,2-yl.

53. A method as in claim 40, wherein said inflammatory response is a response to a physical stimulus.

54. A method as in claim 40, wherein said inflammatory response is a response to a chemical stimulus.

55. A method as in claim 40, wherein said inflammatory response is a response to infection.

56. A method as in claim 40, wherein said inflammatory response is a response to an invasion by a body that is foreign to said subject.

57. A method as in claim 40, wherein said inflammatory response is a response to an immunological stimulus.

58. A method as in claim 40, wherein said inflammatory response is a response to a non-immunological stimulus.

59. A method as in claim 40, wherein said inflammatory response is a response to at least one of the conditions: allergy, asthma, chronic obstructed pulmonary disease (COPD), atherosclerosis, rheumatoid arthritis, multiple sclerosis, and inflammatory bowel disease.

60. A method as in claim 41, wherein said inflammatory bowel disease is at least one of Crohn's disease and ulcerative colitis.

61. A method as in claim 40, wherein said inflammatory response is one of psoriasis, allergic rhinitis, scleroderma, autoimmune thyroid disease, immune-mediated diabetes mellitus, and lupus.

62. A method as in claim 40, wherein said inflammatory response is a response to at least one of the conditions: myasthenia gravis, autoimmune neuropathy.

63. A method as in claim 62, wherein said autoimmune neuropathy is Guillain-Barre neuropathy.

64. A method as in claim 40, wherein said inflammatory response is a response to at least one of autoimmune uveitis, autoimmune hemolytic anemia, pernicious anemia, autoimmune thrombocytopenia, temporal arteritis, anti-phospholipid syndrome, vasculitides.

65. A method as in claim 64, wherein said vasculitides is Wegener's granulomatosis.

66. A method as in claim 40, wherein said inflammatory response is a response to at least one of Behcet's disease, dermatitis herpetiformis, pemphigus vulgaris, vitiligio, primary biliary cirrhosis, autoimmune hepatitis, autoimmune oophoritis, autoimmune orchitis, autoimmune disease of the adrenal gland, polymyositis, dermatomyositis, spondyloarthropathy.

67. A method as in claim 66, wherein said spondyloarthropathy is ankylosing spondylitis.

68. A method as in claim 40, wherein said inflammatory response is a response to Sjogren's syndrome.

69. A method as in claim 40, wherein said inflammatory response is acute inflammation.

70. A method as in claim 40, wherein said inflammatory response is allergic inflammation.

71. A method as in claim 40, wherein said inflammatory response is chronic inflammation.

72. A method for treating or preventing an H₄ receptor-mediated condition in a subject, comprising administering to the subject a pharmaceutical composition that comprises a therapeutically effective amount of at least one of an H₄ receptor modulator of formula (I): 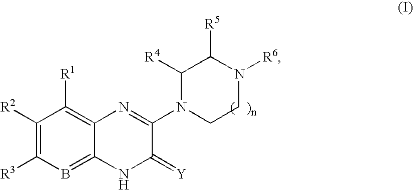
wherein
B is, independently from other member and substituent assignments, N or CR⁷;
Y is, independently from other member and substituent assignments, O, S or NH;
n is, independently from member and substituent assignments, 1 or 2;
each of the substituents R¹⁻³ and R⁷ is, independently from other member and substituent assignments, H, F, Cl, Br, I, C₁₋₄alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of R^{a}, and R b is, independently from other substituent assignments, selected from H, C₁₋₄alkyl, phenyl, benzyl or phenethyl, and wherein any phenyl, alkyl, and cycloalkyl moiety in any of said R¹⁻³, R⁷, R^{a}, and R^{b} is optionally, and independently from other substituent assignments, substituted with 1 to 3 substituents selected from C₁₋₃alkyl, halo, hydroxy, amino, and C₁₋₃alkoxy;
each of R⁴ and R⁵ is, independently from other member and substituent assignments, H or C₁₋₆alkyl;
R⁶ is, independently from other member and substituent assignments, H, C₁₋₆ alkyl, C₃₋₅alkenyl with no sp²-carbon member attached directly to the R⁶-attached nitrogen member, C₃₋₅alkynyl with no sp-carbon member attached directly to the R⁶-attached nitrogen member, CH₂CH₂OH, or —C₁₋₄alkyl-O—C₁₋₄alkyl;
alternatively, R⁶ may be taken together with R⁵, the R⁵-attached carbon member, and the R⁶-attached nitrogen member to form a 5-, 6- or 7-membered heterocyclic ring HetCyc1, wherein said ring HetCyc1 has 0 or 1 additional heteroatoms selected from O, S, >NH or >NC₁₋₆alkyl, and wherein said ring HetCyc1 is substituted with 0, 1, 2 or 3 substituents each selected independently from other substituent assignments from C₁₋₃alkyl, halo, hydroxy, amino, and C₁₋₃alkoxy;
an enantiomer, diastereomer, racemate thereof, and pharmaceutically acceptable salt and ester thereof.

73. A method as in claim 72, wherein wherein B is CR⁷.

74. A method as in claim 72, wherein Y is O.

75. A method as in claim 72, wherein n is 1.

76. A method as in claim 72, wherein each of R¹⁻³ and R⁷ is, independently from other member and substituent assignments, selected from the group consisting of H, —F, —Cl, —Br, —I, —CH₃, —CH₂CH₃, —OCH₃, —OCH₂CH₃, —OCH(CH₃)₂, cyclopropyl, cyclobutyl, cyclopentyl, cyclohexyl, -Ocyclopentyl, -Ocyclohexyl, —CF₃, —OCF₃, —SCF₃, —OH, —NO₂, —NH₂, —NHCH₃, —N(CH₃)₂, —N(CH₂CH₃)₂, —CN and phenyl.

77. A method as in claim 72, wherein R¹⁻³ and R⁷ are independently selected from the group consisting of hydrogen, methyl, trifluoromethyl, methoxy, trifluoromethoxy, nitro, chloro, and fluoro.

78. A method as in claim 72, wherein one or two of R¹⁻³ and R⁷ are not hydrogen.

79. A method as in claim 72, wherein R⁴ and R⁵ are, independently, selected from the group consisting of
a) H, and
b) —CH₃, —CH₂CH₃, —CH₂CH₂CH₃, —CH(CH₃)₂, n-butyl, i-butyl, t-butyl.

80. A method as in claim 72, wherein R⁴ and R⁵ are independently H or —CH₃.

81. A method as in claim 72, wherein R⁶ is selected from the group consisting of
a) H,
b) CH₂CH₂OH, and
c) —CH₃, —CH₂CH₃, —CH₂CH₂CH₃, —CH(CH₃)₂, n-butyl, i-butyl, t-butyl, —CH₂CH₂OCH₃, —CH₂CH₂OCH₂CH₃, —CH₂CH₂OCH₂CH₂CH₃, —CH₂CH₂OCH(CH₃)₂, —CH₂CH₂O-n-butyl, —CH₂CH₂O-1-butyl, and —CH₂CH₂O-t-butyl.

82. A method as in claim 72, wherein R⁶ is selected from the group consisting of H, —CH₃, and —CH₂CH₃.

83. A method as in claim 72, wherein R⁵ taken together with an adjacent R⁵ as well as their carbon and nitrogen of attachment is selected from the group consisting of pyrrolidin-1,2-yl, pyrazolidin-1,5-yl, piperidin-1,2-yl, piperazin-1,2-yl, morpholin-4,5-yl and thiomorpholin-4,5-yl.

84. A method as in claim 72, wherein R⁶ taken together with an adjacent R⁵ as well as their carbon and nitrogen of attachment is pyrrolidin-1,2-yl or piperidin-1,2-yl.

85. A method for modulating an H₄ receptor, comprising exposing an H₄ receptor to modulator comprising at least one of a compound of formula (I): 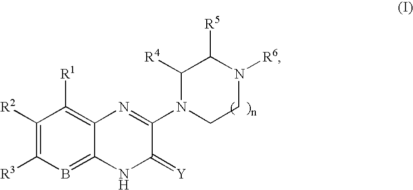
wherein
B is, independently from other member and substituent assignments, N or CR⁷;
Y is, independently from other member and substituent assignments, O, S or NH;
n is, independently from member and substituent assignments, 1 or 2;
each of the substituents R¹⁻³ and R⁷ is, independently from other member and substituent assignments, H, F, Cl, Br, I, C₁₋₄ alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of R^{a}, and R^{b} is, independently from other substituent assignments, selected from H, C₁₋₄alkyl, phenyl, benzyl or phenethyl, and wherein any phenyl, alkyl, and cycloalkyl moiety in any of said R¹⁻³, R⁷, R^{a}, and R^{b}, is optionally, and independently from other substituent assignments, substituted with 1 to 3 substituents selected from C₁₋₃alkyl, halo, hydroxy, amino, and C₁₋₃alkoxy;
each of R⁴ and R⁵ is, independently from other member and substituent assignments, H or C₁₋₆alkyl;
R⁶ is, independently from other member and substituent assignments, H, C₁₋₆ alkyl, C₃₋₅alkenyl with no sp²-carbon member attached directly to the R⁶-attached nitrogen member, C₃₋₅alkynyl with no sp-carbon member attached directly to the R⁶-attached nitrogen member, CH₂CH₂OH, or —Cl₄alkyl-O—C₁₋₄alkyl;
alternatively, R⁵ may be taken together with R⁵, the R⁵-attached carbon member, and the R⁶-attached nitrogen member to form a 5-, 6- or 7-membered heterocyclic ring HetCycl, wherein said ring HetCycl has 0 or 1 additional heteroatoms selected from O, S, >NH or >NC₁₋₆alkyl, and wherein said ring HetCycl is substituted with 0, 1, 2 or 3 substituents each selected independently from other substituent assignments from C₁₋₃alkyl, halo, hydroxy, amino, and C₁₋₃alkoxy;
an enantiomer, diastereomer, racemate thereof, and pharmaceutically acceptable salt and ester thereof.

86. A method as in claim 85, wherein B is CR⁷.

87. A method as in claim 85, wherein Y is O.

88. A method as in claim 85, wherein n is 1.

89. A method as in claim 85, wherein each of R¹⁻³ and R⁷ is, independently from other member and substituent assignments, selected from the group consisting of H, —F, —Cl, —Br, —I, —CH₃, —CH₂CH₃, —OCH₃, —OCH₂CH₃, —OCH(CH₃)₂, cyclopropyl, cyclobutyl, cyclopentyl, cyclohexyl, -Ocyclopentyl, -Ocyclohexyl, —CF₃, —OCF₃, —SCF₃, —OH, —NO₂, —NH₂, —NHCH₃, —N(CH₃)₂, —N(CH₂CH₃)₂, —CN and phenyl.

90. A method as in claim 85, wherein R¹⁻³ and R⁷ are independently selected from the group consisting of hydrogen, methyl, trifluoromethyl, methoxy, trifluoromethoxy, nitro, chloro, and fluoro.

91. A method as in claim 85, wherein one or two of R¹⁻³ and R⁷ are not hydrogen.

92. A method as in claim 85, wherein R⁴ and R⁵ are, independently, selected from the group consisting of
a) H, and
b) —CH₃, —CH₂CH₃, —CH₂CH₂CH₃, —CH(CH₃)₂, n-butyl, i-butyl, t-butyl.

93. A method as in claim 85, wherein R⁴ and R⁵ are independently H or —CH₃.

94. A method as in claim 85, wherein R is selected from the group consisting of
a) H,
b) CH₂CH₂OH, and
c) —CH₃, —CH₂CH₃, —CH₂CH₂CH₃, —CH(CH₃)₂, n-butyl, i-butyl, t-butyl, —CH₂CH₂OCH₃, —CH₂CH₂OCH₂CH₃, —CH₂CH₂OCH₂CH₂CH₃, —CH₂CH₂OCH(CH₃)₂, —CH₂CH₂O-n-butyl, —CH₂CH₂O-1-butyl, and —CH₂CH₂O-t-butyl.

95. A method as in claim 85, wherein R⁶ is selected from the group consisting of H, —CH₃, and —CH₂CH₃.

96. A method as in claim 85, wherein R⁶ taken together with an adjacent R⁵ as well as their carbon and nitrogen of attachment is selected from the group consisting of pyrrolidin-1,2-yl, pyrazolidin-1,5-yl, piperidin-1,2-yl, piperazin-1,2-yl, morpholin-4,5-yl and thiomorpholin-4,5-yl.

97. A method as in claim 85, wherein R⁶ taken together with an adjacent R⁵ as well as their carbon and nitrogen of attachment is pyrrolidin-1,2-yl or piperidin-1,2-yl.

98. A method as in claim 85, wherein said modulator is a receptor antagonist.

99. A method as in claim 85, wherein said modulator is a receptor partial agonist.

100. A method for inhibiting leukocyte recruitment in a subject, comprising administering to the subject a pharmaceutical composition that comprises a therapeutically effective amount of at least one of a leukocyte recruitment inhibitor of formula (I): 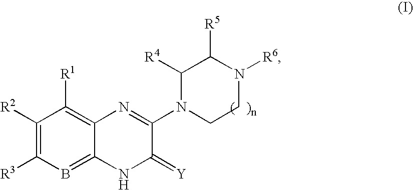
wherein
B is, independently from other member and substituent assignments, N or CR⁷;
Y is, independently from other member and substituent assignments, O, S or NH;
n is, independently from member and substituent assignments, 1 or 2;
each of the substituents R¹⁻³ and R⁷ is, independently from other member and substituent assignments, H, F, Cl, Br, I, C₁₋₄ alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of R^{a}, and R^{b} is, independently from other substituent assignments, selected from H, C₁₋₄alkyl, phenyl, benzyl or phenethyl, and wherein any phenyl, alkyl, and cycloalkyl moiety in any of said R-3, R⁷, R^{a}, and Rb, is optionally, and independently from other substituent assignments, substituted with 1 to 3 substituents selected from C₁₋₃alkyl, halo, hydroxy, amino, and C₁₋₃alkoxy;
each of R⁴ and R⁵ is, independently from other member and substituent assignments, H or C₁₋₆alkyl;
R⁶ is, independently from other member and substituent assignments, H, C₁₋₆ alkyl, C₃₋₅alkenyl with no sp²-carbon member attached directly to the R⁶-attached nitrogen member, C₃₋₅alkynyl with no sp-carbon member attached directly to the R⁵-attached nitrogen member, CH₂CH₂OH, or —C₁₋₄alkyl-O—C₁₋₄alkyl;
alternatively, R may be taken together with R⁵, the R⁵-attached carbon member, and the R⁶-attached nitrogen member to form a 5-, 6- or 7-membered heterocyclic ring HetCycl, wherein said ring HetCycl has 0 or 1 additional heteroatoms selected from O, S, >NH or >NC₁₋₆alkyl, and wherein said ring HetCycl is substituted with 0, 1, 2 or 3 substituents each selected independently from other substituent assignments from C₁₋₃alkyl, halo, hydroxy, amino, and C₁₋₃alkoxy;
an enantiomer, diastereomer, racemate thereof, and pharmaceutically acceptable salt and ester thereof.

101. A method as in claim 100, wherein B is CR⁷.

102. A method as in claim 100, wherein Y is O.

103. A method as in claim 100, wherein n is 1.

104. A method as in claim 100, wherein each of R¹⁻³ and R⁷ is, independently from other member and substituent assignments, selected from the group consisting of H, —F, —Cl, —Br, —I, —CH₃, —CH₂CH₃, —OCH₃, —OCH₂CH₃, —OCH(CH₃)₂, cyclopropyl, cyclobutyl, cyclopentyl, cyclohexyl, —Ocyclopentyl, —Ocyclohexyl, —CF₃, —OCF₃, —SCF₃, —OH, —NO₂, —NH₂, —NHCH₃, —N(CH₃)₂, —N(CH₂CH₃)₂, —CN and phenyl.

105. A method as in claim 100, wherein R¹⁻³ and R⁷ are independently selected from the group consisting of hydrogen, methyl, trifluoromethyl, methoxy, trifluoromethoxy, nitro, chloro, and fluoro.

106. A method as in claim 100, wherein one or two of R¹⁻³ and R⁷ are not hydrogen.

107. A method as in claim 100, wherein R⁴ and R⁵ are, independently, selected from the group consisting of
a) H, and
b) —CH₃, —CH₂CH₃, —CH₂CH₂CH₃, —CH(CH₃)₂, n-butyl, i-butyl, t-butyl.

108. A method as in claim 100, wherein R⁴ and R⁵ are independently H or —CH₃.

109. A method as in claim 100, wherein R is selected from the group consisting of
a) H,
b) CH₂CH₂OH, and
c) —CH₃, —CH₂CH₃, —CH₂CH₂CH₃, —CH(CH₃)₂, n-butyl, i-butyl, t-butyl, —CH₂CH₂OCH₃, —CH₂CH₂OCH₂CH₃, —CH₂CH₂OCH₂CH₂CH₃, —CH₂CH₂OCH(CH₃)₂, —CH₂CH₂O-n-butyl, —CH₂CH₂O-1-butyl, and —CH₂CH₂O-t-butyl.

110. A method as in claim 100, wherein R⁶ is selected from the group consisting of H, —CH₃, and —CH₂CH₃.

111. A method as in claim 100, wherein R⁶ taken together with an adjacent R⁵ as well as their carbon and nitrogen of attachment is selected from the group consisting of pyrrolidin-1,2-yl, pyrazolidin-1,5-yl, piperidin-1,2-yl, piperazin-1,2-yl, morpholin-4,5-yl and thiomorpholin-4,5-yl.

112. A method as in claim 100, wherein R⁶ taken together with an adjacent R⁵ as well as their carbon and nitrogen of attachment is pyrrolidin-1,2-yl or piperidin-1,2-yl.

113. A method for making a quinoxaline compound, comprising reacting a diamino compound of formula (III) with an ester of formula (IV), 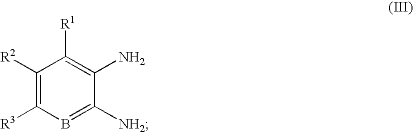
(RO)₃CCO₂R (IV)
wherein
B is, independently from other member and substituent assignments, N or CR⁷;
each of the substituents R¹⁻³ and R⁷ is, independently from other member and substituent assignments, H, F, Cl, Br, I, C₁₋₄ alkyl, C₂₋₅alkenyl, C₂₋₅alkynyl, C₁₋₄alkoxy, C₁₋₄alkylthio-, —C₃₋₆cycloalkyl, —OC₃₋₆cycloalkyl, —OCH₂Ph, —CF₃, —OCF₃, —SCF₃, —OH, nitro, —NR^{a}R^{b}, cyano, phenyl, wherein each of
R^{a}, and R^{b} is, independently from other substituent assignments, selected from H, C₁₋₄alkyl, phenyl, benzyl or phenethyl, and wherein any phenyl, alkyl, and cycloalkyl moiety in any of said R¹⁻³, R⁷, R^{a}, and R^{b}, is optionally, and independently from other substituent assignments, substituted with 1 to 3 substituents selected from C₁₋₃alkyl, halo, hydroxy, amino, and C₁₋₃alkoxy; and
R is one of C₁₋₆alkyl and benzyl.

114. A method as in claim 113, wherein said quinoxaline compound is at least one of a compound of formula (I): 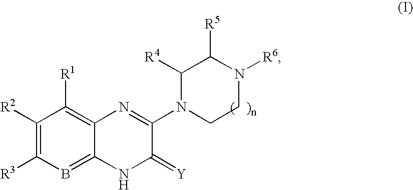
wherein
Y is, independently from other member and substituent assignments, O, S or NH;
n is, independently from member and substituent assignments, 1 or 2;
each of R⁴ and R⁵ is, independently from other member and substituent assignments, H or C₁₋₆alkyl; and
R⁶ is, independently from other member and substituent assignments, H, C₁₋₆ alkyl, C₃₋₅alkenyl with no sp²-carbon member attached directly to the R⁶-attached nitrogen member, C₃₋₅alkynyl with no sp-carbon member attached directly to the R⁶-attached nitrogen member, CH₂CH₂OH, or —C₁₋₄alkyl-O—C₁₋₄alkyl;
alternatively, R⁵ may be taken together with R⁵, the R⁵-attached carbon member, and the R⁶-attached nitrogen member to form a 5-, 6- or 7-membered heterocyclic ring HetCyc1, wherein said ring HetCyc1 has 0 or 1 additional heteroatoms selected from O, S, >NH or >NC₁₋₆alkyl, and wherein said ring HetCyc1 is substituted with 0, 1, 2 or 3 substituents each selected independently from other substituent assignments from C₁₋₃alkyl, halo, hydroxy, amino, and C₁₋₃alkoxy.

115. A method as in claim 113, wherein R is one of methyl and ethyl.

116. A method as in claim 113, wherein said reacting is carried out at a temperature of at least about 40° C.

117. A method as in claim 116, wherein said reacting is carried out at a temperature of about 100° C.

118. A method as in claim 113, wherein said reacting is performed in a solvent whose boiling point is at least about 100° C.

119. A method as in claim 113, wherein said reacting is preferably performed in toluene.

120. A method as in claim 113, wherein said reacting further comprises incorporating into the reaction medium a Lewis acid catalyst or a protic acid catalyst.

121. A method as in claim 120, wherein said Lewis acid catalyst is a lanthanide triflate.

122. A method as in claim 120, wherein said Lewis acid catalyst is one of ytterbium triflate, scandium triflate, zinc chloride, copper triflate, or mixtures thereof.

123. A method as in claim 122, wherein said Lewis acid catalyst is ytterbium triflate.

124. A method as in claim 120, wherein said protic acid catalyst is p-toluenesulfonic acid.

125. A method as in claim 124, wherein said protic acid catalyst is used under Dean-Stark conditions.

126. A method as in claim 120, further comprising an addition-elimination reaction of a secondary amine of formula (VI) with a compound of formula (V) that is formed in said reacting of said diamino compound with said ester, 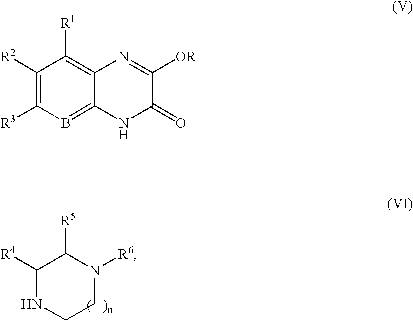
wherein R is such that the group OR in compound of formula (VI) is a suitable leaving group in said addition-elimination reaction.

127. A method as in claim 126, wherein said secondary amine is a piperazine derivative or a homopiperazine derivative.

128. A method as in claim 126, wherein said addition-elimination reaction is performed at a temperature of at least about 40° C.

129. A method as in claim 128, wherein said temperature is at least about 100° C.

130. A method as in claim 128, wherein said temperature is a temperature of about 175° C.

131. A method as in claim 126, wherein said performing said addition-elimination reaction further comprises incorporating into the reaction medium of a catalyst.

132. A method as in claim 131, wherein said catalyst is hydroxypyridine.
